(19) United States
(12) Patent Application Publication
(10) Pub. No.: US 2024/0277818 A1
(43) Pub. Date: Aug. 22, 2024

(54) COMPOSITION INCLUDING A METAL-ORGANIC FRAMEWORK (MOF) NANOPARTICLE FOR ORAL PROTEIN ADMINISTRATION

(71) Applicants: UNIVERSITY OF NORTH TEXAS, Dallas, TX (US); WUHAN UNIVERSITY, Wuhan (CN)

(72) Inventors: Shengqian MA, Dallas, TX (US); Jian TIAN, Dallas, TX (US)

(21) Appl. No.: 18/584,761

(22) Filed: Feb. 22, 2024

Related U.S. Application Data
(60) Provisional application No. 63/486,296, filed on Feb. 22, 2023.
(60) Provisional application No. 63/495,472, filed on Apr. 11, 2023.

(51) Int. Cl. A61K 38/28; A61K 9/00; A61K 47/55; A61K 47/64; A61K 47/69

(52) U.S. Cl. CPC A61K 38/28; A61K 9/0053; A61K 47/551; A61K 47/644; A61K 47/6935

(57) ABSTRACT

A composition can comprise a metal-organic framework (MOF) nanoparticle for oral protein administration, wherein the MOF comprises a nano-scale acid-resistant MOF; a small molecular protein/polypeptide drug and a targeting molecule, wherein the MOF is loaded with the small molecular protein/polypeptide; and targeting molecules, wherein a surface of the MOF is coated with the targeting molecules.

### CROSS-REFERENCES TO RELATED APPLICATIONS

This application claims the benefit of U.S. Provisional Application No. 63/486,296, filed on Feb. 22, 2023, and entitled “FUNCTIONALIZED METAL-ORGANIC FRAMEWORK NANOPARTICLES FOR ORAL INSULIN DELIVERY,” and U.S. Provisional Application No. 63/495,472, filed on Apr. 11, 2023, and entitled “EFFICIENT ORAL INSULIN DELIVERY WITH SUSTAINED-RELEASE BY FOLATE-CONJUGATEDMETAL-ORGANIC FRAMEWORK NANOPARTICLES,” which are incorporated herein by reference in their entirety for all purposes.

### STATEMENT REGARDING FEDERALLY SPONSORED RESEARCH OR DEVELOPMENT

None.

### BACKGROUND

Oral protein delivery is considered a cutting-edge technology to improve patients' quality of life, offering superior patient compliance and convenience compared to injections. Oral protein/peptide delivery systems have drawn worldwide attention for their ability to improve patient compliance and clinical outcomes by alleviating the fear associated with constant injections. Unfortunately, their clinical translation has been severely limited by poor intestinal permeability, acid instability, and a short half-life of protein/peptide. As such, oral protein formulation has stagnated due to the instability and inefficient penetration of protein in the gastrointestinal tract.

As an example, diabetes is a chronic metabolic disease that affects more than 470 million people around the world. Because of its high prevalence, disability, and fatality rate, diabetes has become a serious global health problem. The treatment of type I diabetes or advanced type II diabetes relies on routinely subcutaneous insulin (INS) injections. Still, long-term INS subcutaneous injections are accompanied by various side effects, resulting in poor patient compliance. Hence, the development of non-invasive INS delivery methods is in urgent need. Among various non-invasive methods of administration, oral administration has been the most widely used way due to its painlessness and convenience. Nevertheless, barely any protein pharmaceutical preparations have been approved for oral administration in clinic practice, primarily due to the formidable physiological barrier in the gastrointestinal (GI) tract. These barriers mainly include harsh acids and proteases in the stomach, the impermeable mucus layer, and the tightly connected intestinal epithelial cell layer in the intestine, which severely hampered the absorption efficiency of active protein formulations. Above all, how to maintain high protein activity and allow proteins to penetrate through the epithelial cell layer into the bloodstream are considered the main challenges of any oral drug formulation.

To overcome the above-mentioned absorption barriers, scientists have developed numerous nanoparticle (NP) platforms for oral protein delivery, such as liposomes, polymeric NPs, and silica NPs. However, the capability of these NPs for practical protein delivery applications still remains limited, suffering from inefficient protein loading and protection, low intestinal permeation, uncontrollable release, or complicated formulation strategies. In recent years, employing enteric materials and penetration enhancers have received significant attention to enhance the efficacy of protein delivery. However, enteric coating involves additional excipients and formulation steps. Penetration enhancers are associated with a risk of destroying the tight junctions of the intestinal epithelial cell layer, which may lead to bacterial infection. Therefore, it is vital to achieve effective protein absorption without destroying the tight junctions of the intestinal epithelial cell layer. From this perspective, some progress has been made in the design of macromolecular protein drug carriers utilizing various specific receptors highly expressed on the intestinal epithelial cell membrane, such as transferrin receptors (TfR) and FcRn receptors. For instance, transferrin (Tf) widely exists in the human body, which guides iron absorption and transportation by binding to TfR on epithelial cells. Notably, Tf/TfR-mediated targeted protein delivery systems can produce exciting results in cancer, central nervous system diseases, and oral administration. The Tf-INS conjugate increases the permeability of INS across the epithelial barrier by receptor-mediated transcytosis. Nevertheless, the fate of Tf in cells after transcytosis is going to lysosomes, where the abundant proteases could damage protein structure, leading to limited bioavailability. Tf-coated polymeric NPs for oral INS delivery have been developed, in which the surface-loaded Tf could enhance their transepithelial transport via receptor-mediated transcytosis. However, these NPs demonstrate unfavorable INS release before the transepithelial transport that restricted their pharmacological availability (about 7.8%). Therefore, the development of functional nanosystems to protect the protein from the harsh gastric environment while improving the penetration efficacy through the intestine and realizing controlled release is of great significance for oral protein delivery.

### BRIEF DESCRIPTION OF THE DRAWINGS

For a more complete understanding of the present disclosure and the advantages thereof, reference is now made to the following brief description, taken in connection with the accompanying drawings and detailed description, wherein like reference numerals represent like parts.

FIG. 1 is the protein loading of the metal-organic framework nanoparticles prepared in example 1.

FIG. 2 is a transmission electron microscope image (scale: 100 nm) of the metal-organic framework nanoparticles prepared in example 1.

FIG. 3 is the effect of insulin released from equal amounts of free insulin and protein oral nanoparticles on blood glucose in normal rats in example 2.

FIG. 4 is the protein degradation curves of I @ U and T @ I @ U from example 3 after incubation with trypsin in HBSS buffer at 37° C. for various time points.

FIG. 5 is the in vitro release kinetics results for I @ U and T @ I @ U of example 4 under different pH, salt conditions.

FIG. 6 is the results of in vitro permeation of free insulin, I @ U and T @ I @ U in different intestinal segments (A. duodenum. B. jejunum. C. ileum) from example 5.

FIG. 7 is the blood glucose level change and serum insulin concentration (C) of diabetic BALB/C mice (A) and SD rats (B) following oral administration of free insulin, I @ U and T @ I @ U in example 6.

FIG. 8 is a schematic representation of FA-modified Zr-based MOF for sustained oral delivery of insulin. (a) The synthesis process of FA-modified Zr-based MOF delivery system. (b) Schematic illustration of the sustained release mechanism of FA-modified Zr-based MOF. (c) Schematic illustration of the FA-modified Zr-based MOF selective endocytosis pathways. In the presence of low expression of PCFT in normal animals, FA-modified Zr MOFs were mainly internalized by a clathrin-mediated pathway and subsequently digested by lysosomes. In the case of high expression of PCFT in diabetic animals, the MOF is endocytosed through the pathway mediated by caveolae and thus exocytosed in a complete form to achieve efficient transcellular transport efficiency.

FIG. 9 is the preparation and characterizations of PCN-777-based nanosystems. (a) The synthesis process of sustained release MOF system. (b) TEM images of PCN-777-based nanosystems. Scale bar equals 200 nm. (c) Size distributions and zeta potential of PCN-777-based nanosystems. (d) Emission spectra of different preparations with excitation at 280 nm. (e) Emission spectra of different preparations with excitation at 560 nm. (f) PXRD patterns of as-synthesized PCN-777-based nanosystems. (g) The EDS elemental mapping analyses of I@F-P NPs. Scale bar equals 50 nm.

FIG. 10 is the in vitro release performance of the FA-modified PCN-777 nanosystem. (a) Comparison of the INS loading capacity of PCN-777-based nanosystems with different feeding ratios (n equals three independent samples, mean plus or minus standard deviation (SD)). (b) The enzymatic degradation profiles from different preparations after incubation with trypsin-buffer at 37° C. for various time points (n equals three independent samples, mean plus or minus SD). (c) Blood glucose response of normal rats to free INS and INS released from I@F-P NPs (n equals three biologically independent animals, mean plus or minus SD). (d) The in vitro release kinetics of I@P, and I@F-P with different feed ratios under phosphate conditions (n equals three independent samples, mean plus or minus SD). (e) Continuous release of INS from I@P, and I@F-P under different pH environments (n equals three independent samples, mean plus or minus SD). (f) The hemolysis analysis of I@F-P (n equals three independent samples, mean plus or minus SD). (g) The changes of morphology of I@P and I@F-P immersed in PBS for 48 h. Scale bar equals 200 nm.

FIG. 11 is the biocompatibility of PCN-777-based nanosystems. (a) The in vitro cell viability test of PCN-777-based nanosystems for Caco-2 cells, CT-26 cells, and L02 cells (n equals five biological independent samples, mean plus or minus SD). (b) The live/dead imaging of PCN-777-based nanosystems. Scale bar equals 100 m. (c) Analysis of blood biochemical indicators two weeks after PCN-777-based nanosystem administration (n equals five biologically independent animals, mean plus or minus SD). Abbreviations: aspartate aminotransferase, AST; alanine aminotransferase, ALT; alkaline phosphatase, ALP; albumin, ALB; total bilirubin, T-bil; uric acid, UA; creatinine, CR; bureau nitrogen, BUN; lactate dehydrogenase, LDH; creatine kinase, CK; creatine kinase isoenzyme, CK-MB.

FIG. 12 is the PCFT-mediated selective endocytosis of I@F-P. (a) CLSM 3D images and (b) fluorescence quantitative determination in different distant of RITC labeled Free INS, I@P, and I@F-P incubated with different PCFT expression levels of Caco-2 cell monolayers for 4 h. (c) CLSM images of the endocytosis mechanisms of I@F-P (red) in Caco-2 cells (blue) pretreated with different inhibitors. Scale bar equals 15 m. Abbreviations: CPZ, chlorpromazine; LV, lovastatin; M-PCD, methyl-b-cyclodextrin; CYD, cytochalasin D. (d) Quantitative determination of I@F-P cellular uptake amounts for different inhibitors treatments at 4 h. (e) The co-localization images of Golgi (green) and I@F-P (red) with different feed ratios at 4 hours. Scale bar equals 10 m. (f) The co-localization images of the lysosome (green) and I@F-P (red) with different feed ratios at 4 hours. Scale bar equals 10 μm.

FIG. 13 is the transmembrane transport of I@F-P in the PCFT high expression and PCFT low expression Caco-2 cell monolayer model. (a) Effects of different formulations on TEER of Caco-2 cell monolayers (n equals three biologically independent samples, mean plus or minus SD). The (b) cumulative transportation and (c) apparent permeability coefficient (Papp) of INS from different formulations across Caco-2 cell monolayer in 48 h (n equals three biologically independent samples, mean plus or minus SD). (d) Lysosomal degradation of INS from different formulations measured after incubating the cell monolayers for 48 h (n equals three biologically independent samples, mean plus or minus SD). (e) Intracellular transfer of different INS formulations visualized by CLSM. Caco-2 cells in batch 1 were cultured in a medium that contained different INS formulations for 12 h. Then, the culture medium for batch 1 was refreshed and fresh cells in batch 2 for 12 h. The above step was repeated to obtain batch 3. Scale bar equals 10 μm.

FIG. 14 is the Enhanced and prolonged hypoglycemic effect validation of FA-modified PCN-777 nanosystem in animals. (a) Cartoon representation of the oral administration of the FA-modified PCN-777 nanosystem to animals. (b) TEM image of epithelial tissues collected 4 h after the oral administration of PCN-777-based nanosystem. Scale bar equals 2 μm. (c) Fluorescence distribution and intensity of the RITC-INS in vivo at different time intervals after the oral INS administration from different formulations. (d) Ex vivo fluorescence images of the intestine, and images of the heart, liver, spleen, lung, and kidney (from top to bottom) of the mice after the oral INS administration for 12 h. (e) Images of sectioned intestine tissues of rats after oral administration of different RITC-INS formulations for 12 h. Scale bar equals 200 m. (f) Blood glucose levels of initial vs. time profiles of the diabetic and normal SD rats following the oral administration of I@F-P, I@P, and Free INS, as well as subcutaneous injection of Free INS (n equals five biologically independent animals, mean plus or minus SD). (g) The serum INS levels vs. time profiles of the diabetic and normal SD rats following the same formulations in (f) (n equals five biologically independent animals, mean plus or minus SD). (h) Time-course profiles of blood glucose levels for from the diabetic and normal New Zealand rabbits following the oral administration of I@F-P and Free INS (n equals three biologically independent animals, mean plus or minus SD). The rabbits were fed at the time points marked by arrows.

FIG. 15 is the thermogravimetric analysis of each PCN-777-based nanosystems.

FIG. 16 is the in vitro release kinetics of I@P, and I@F-P under FaSSGF and FaSSIF (n equals three biologically independent samples, mean plus or minus SD).

FIG. 17 depicts a table of kinetic parameters of fitting release curve (n equals three biologically independent samples, mean plus or minus SD).

FIG. 18 depicts a table of blood routine analysis of BALB/C mice two weeks after PCN-777-based nanosystem administration (n equals five biologically independent animals, mean plus or minus SD).

FIG. 19 depicts H&E staining of harvested heart, liver, spleen, lung, kidney, and small intestine after different treatments.

FIG. 20 is a quantitative cellular uptake amounts of prepared treatments at 4 h.

FIG. 21 is the internalization of different PCN preparations in Caco-2 cells with high expression levels of PCFT. Scale bar equals 10 μm.

FIG. 22 is cellular uptake images of I@F-P in both high and low PCFT expression Caco-2 cells at various time points. Scale bar equals 15 μm.

FIG. 23 is CLSM images depicting the internalization of I@F-P (red) in Caco-2 cells (blue) pretreated with various inhibitors. Scale bar equals 15 μm.

FIG. 24 is (a) schematic diagram of I@F-P crossing the Caco-2 monolayer membrane in a transwell experiment. (b) Specimen photographs for fluorescence images of RITC labeled INS, I@P, and I@F-P across the apical and basolateral sides of Caco-2 cell monolayers at high or low expression levels of PCFT. (c) TEM images of basolateral samples after 24 hours of incubation with different formulations. Scale bar equals 200 nm.

FIG. 25 depicts a table of pharmacokinetic parameters of INS in diabetic rats (n equals five biologically independent animals, mean plus or minus SD).

FIG. 26 is the changes of morphology of T@I@U with the Tf amounts increasing. Scale bar equals 50 nm.

FIG. 27 is the UV-vis absorption spectra of RITC-INS, RITC-INS@U, and UiO-68-NH2. The asterisk symbol represented at 280 nm indicates the characteristic absorption peak of INS.

FIG. 28 is (A) the CLSM images and (B) the inverted fluorescence microscope images of RITC-labeled INS@5 μm UiO-68-NH₂ crystals, indicating the distribution of insulin in the UiO-68-NH₂ crystals. Scale bar equals 1 μm.

FIG. 29 is the zeta potential of INS, UiO-68-NH₂, and I@U at the acidic condition of the loading process (pH equals 4).

FIG. 30 is the changes of protein loading content of I@U and T@I@U as the ratio of INS: UiO-68-NH₂ increasing (n equals three independent samples, mean plus or minus SD).

FIG. 31 is the thermogravimetric analysis of UiO-68-NH₂, I@U, and T@I@U.

FIG. 32 is the size stability of UiO-68-NH₂-based nanosystems in PBS and DMEM (10% FBS) incubated at 4° C. for 24 h.

FIG. 33 is the changes of morphology of I@U and T@I@U immersed in PBS for 4 h. Scale bar equals 100 nm.

FIG. 34 is the in vitro release kinetics of I@U, and T@I@U in ddH₂O and PBS (pH equals 7.4).

FIG. 35 is the biocompatibility of UiO-68-NH₂-based nanosystem. (A) The hemolysis analyzes of T@I@U. The cell viability test of UiO-68-NH₂-based nanosystems in vitro for Caco-2 cells (B), CT-26 cells (C), and L02 cells (D) (n equals five biological independent samples, mean plus or minus SD). (E) The live/dead imaging of UiO-68-NH₂-based nanosystems.

FIG. 36 is the H&E staining of harvested heart, liver, spleen, lung, kidney, and small intestine after various treatments.

FIG. 37 is the co-localization graphs of lysosome (green) and T@I@U (red) at different time points.

FIG. 38 is the lysosomal degradation of INS from different formulations measured after incubating the cell monolayers for 2 h (n equals three biological independent samples, mean plus or minus SD).

FIG. 39 depicts a table of insulin loading content in different types of materials.

FIG. 40 depicts a table of pharmacokinetic parameters of INS in diabetic rats after subcutaneous injection of free INS (5 IU/kg) and oral administration of free INS (20 IU/kg), I@U (20 IU/kg), and T@I@U (20 IU/kg) (n equals three).

### DETAILED DESCRIPTION

It should be understood at the outset that although an illustrative implementation of one or more embodiments is provided below, the disclosed systems and/or methods may be implemented using any number of techniques, whether currently known or in existence. The disclosure should in no way be limited to the illustrative implementations, drawings, and techniques below, including the exemplary designs and implementations illustrated and described herein, but may be modified within the scope of the appended claims along with their full scope of equivalents.

As used herein, the term “and/or” can mean one, some, or all elements depicted in a list. As an example, “A and/or B” can mean A, B, or a combination of A and B. Furthermore, the symbol “/” between elements A and B, e.g., “A/B”, may also mean “A and/or B”.

Among numerous nanomaterials, nanoscale metal-organic frameworks (nMOFs) have emerged as promising candidates for drug delivery. The nMOFs are a relatively new class of hybrid porous materials composed of metal ions or clusters bridged by organic ligands, which have ordered pore structure, large specific surface area, and abundant modification sites. Therefore, nMOFs hold great potential as drug transport carriers because they can achieve extremely high drug loading capacity and connect with different molecules as needed to fulfill various functions. Moreover, the reversible coordination between ligands and metal ions inside endow the nMOFs with the characteristics of controlled releasing, biodegradability, low toxicity, and good biocompatibility. Recently, a mesoporous Zr-based MOF (NU-1000) as an INS carrier has been reported, protecting INS from simulated harsh stomach environments and controlling INS release under physiological conditions. Although the preliminary in vitro release test achieved favorable results, the problem of low protein permeability in the intestinal epithelial cell layer remains to be solved, and there is still a lack of in vivo study evidence to prove whether effective INS absorption can be achieved. As understood, no research about MOF-based nanosystems protecting INS from a harsh environment while improving the INS penetration efficacy has been reported to achieve a superior hypoglycemic effect ultimately.

The present compositions and methods disclose a metal organic framework nano-particle for oral protein administration and a preparation method thereof. The metal organic framework nano-particle provided by the present disclosure can promote the oral absorption of protein/polypeptide with the molecular weight of up to about 10000 Daltons. The metal organic framework nano-particle is prepared by loading a nano-scale acid-resistant metal organic framework with a small-molecule protein/polypeptide drug through hydrophobic interaction and modifying a target molecule on the surface. The porous acid-resistant metal organic framework shows high drug-loading capacity for protein, strong protection, and good biocompatibility and slow and controlled release kinetics in vivo. The targeting molecule can target the receptor on the intestinal epithelial cell membrane, overcome the problem of extremely low permeation efficiency of the protein in the intestinal epithelial cell layer, and improve the oral bioavailability of the protein. The preparation method is simple and convenient, has low production cost, and is expected to realize painless, controlled release and convenient protein oral administration.

The polypeptide and protein medicine is polypeptide and protein biological medicine for preventing, treating and diagnosing and has the advantages of high activity, high specificity, low toxicity, clear biological function, etc. The administration route is more critical due to the complex preparation process, low delivery efficiency, poor bioavailability and the like of polypeptide and protein medicines. However, the protein drug membrane has poor permeability and short half-life, and is easy to be damaged in strong acid, strong alkali and environment rich in protease. Thus, subcutaneous injection is the primary mode of administration of proteins. The long-term frequent protein injection can bring considerable pressure and pain to the mind and body of a patient, and the injection part can have serious side effects of skin red and swollen, itching, induration, infection, subcutaneous lipoatrophy, fibrosis hyperplasia and the like. Therefore, development of a non-invasive protein delivery method is urgently required. Among the many non-invasive modes of administration, oral administration is the most widely used mode because of its painlessness and convenience. However, due to the three major physiological barriers present in the gastrointestinal tract, the strong acids and proteases in the stomach, the mucus layer in the intestinal tract and the tightly associated intestinal epithelial cell layer, the oral bioavailability of proteins is extremely low and few pharmaceutical preparations of any protein are approved for oral administration in clinical practice.

In order to overcome the above-mentioned oral absorption barrier of macromolecules and improve the oral bioavailability of proteins, various platforms such as permeation enhancers, enzyme inhibitors, nano-formulations, etc. have been tried to be developed and applied. However, penetration enhancers risk disrupting the tight junctions of the intestinal epithelial cell layer, possibly leading to bacterial infection. Enzyme inhibitors may seriously affect the digestive absorption of proteins and may cause adverse symptoms such as nausea and vomiting. However, most of the nano-preparations (such as liposome, polymer nanoparticle and silica nanoparticle) still have limited capability in practical protein delivery applications, and have the problems of low drug loading, low protein protection efficiency, low intestinal permeability, uncontrollable drug release or complex formulation synthesis strategy, and the like. Therefore, it is important to develop a nano-platform that does not disrupt the intestinal epithelial cell layer tight junctions and enables efficient oral absorption of proteins.

Among the existing numerous nanomaterials, the nanoscale metal-organic framework (nMOF) is a novel porous solid material formed by self-assembly of metal-containing nodes and organic ligands through coordination connection. The nMOF has great potential as a drug delivery carrier: (1) the specific surface area is high, the pore size is large, a large amount of drugs or biological molecules can be loaded, and the carrier is an ideal drug transport carrier; (2) ligands and metal sites in the MOFs can be used as reaction sites, modification is easy to carry out, and the MOFs can be connected with different molecules according to requirements to realize different functions, for example, active targeting can be realized by modifying targeting peptides or targeting molecules; (3) the adjustability of the composition and structure of the MOFs is beneficial to controllably synthesizing the MOFs with different compositions, functions and sizes; (4) under different physiological environments, the MOFs can generate reversible coordination between ligands and metal ions, so that the MOFs have controlled release, biodegradability, low toxicity and good biocompatibility. A method of using zirconium-based MOF as an oral carrier for insulin, and in vitro experimental results shows that the method can protect insulin from strong acid and enzymatic environments and control the release of insulin under physiological conditions (phosphate environment). However, this study does not solve the problem of the very low permeation efficiency of proteins in the intestinal epithelial cell layer.

In order to solve the problem that the protein has extremely low permeation efficiency in the intestinal epithelial cell layer, researches for improving the protein delivery efficiency by using permeation enhancers and the like have received great attention. However, penetration enhancers risk disrupting the tight junctions of the intestinal epithelial cell layer, resulting in bacterial infection. Therefore, achieving efficient protein absorption without disrupting the tight junctions of the intestinal epithelial cell layer is desired. In recent years, some progress has been made in the research of designing macromolecular protein drug carriers using various specific receptors (e.g., transferrin receptor and FcRn receptor) highly expressed on the intestinal epithelial cell membrane. For example, transferrin is widely present in the human body and directs the absorption and transport of iron by binding to transferrin receptors on epithelial cells. Moreover, the study of transferrin/transferrin receptor mediated targeted protein delivery systems has produced exciting results in cancer, central nervous system diseases, and oral administration. It has been reported that transferrin-insulin conjugates increase the penetration efficiency of insulin across epithelial barriers by receptor-mediated endocytosis. Transferrin-conjugated insulin, however, is endocytosed before entering the lysosome, where abundant proteases may disrupt the protein structure, resulting in limited bioavailability. Therefore, the development of functional nanosystems to protect proteins from the harsh gastric environment, while increasing the efficiency of permeation through the intestinal tract and achieving controlled release, is of great importance for oral protein delivery.

The present compositions and methods aims to overcome the defects of low oral bioavailability of protein, complex preparation process of a product and the like in the prior art, and provides a metal organic framework nano-particle for oral protein administration and a preparation method thereof. The metal organic framework nano-particles for oral protein administration can protect protein from being damaged by gastric acid and digestive enzyme in gastrointestinal tracts, can accelerate the absorption of protein medicines by intestinal epithelial cells under the condition of not damaging the tight connection of intestinal tracts, and can achieve the aim of high-efficiency oral absorption of the protein.

In some aspects, a metal organic framework nanoparticle for oral protein administration is a metal organic framework nanoparticle loaded with small molecule proteins/polypeptides, and can promote oral absorption of proteins with molecular weight of up to 10,000 Dalton. The metal organic framework nano-particle mainly comprises a nano acid-resistant metal organic framework, a therapeutic micromolecule protein/polypeptide drug and a targeting molecule, wherein the acid-resistant metal organic framework is a carrier of the micromolecule protein/polypeptide, is loaded with the therapeutic micromolecule protein/polypeptide and is coated with the targeting molecule on the surface. The targeting molecule can target a receptor on an intestinal epithelial cell membrane, so that effective uptake of nanoparticles is realized, and the problem of extremely low permeation efficiency of protein in the intestinal epithelial cell layer is solved. The acid-resistant metal organic framework, the small molecular protein/polypeptide and the targeting molecule are subjected to hydrophobic interaction, so that the acid-resistant metal organic framework encapsulates the small molecular protein/polypeptide, and the targeting molecule is modified on the surface of the acid-resistant metal organic framework to form stable nanoparticles.

The acid-resistant metal organic framework can include, but is not limited to, UiO-68-NH₂, PCN-222, and/or PCN-224.

The small molecular protein/polypeptide can be protein/polypeptide with a molecular weight of less than about 10,000, about 9,000, about 8,000, about 7,000, about 6,000, or about 5,000 Daltons. Some example can include, but are not limited to, insulin, salmon calcitonin, glucagon, interferon, growth hormone, and the like.

The targeting molecule can target receptors on intestinal epithelial cell membranes, so that effective uptake of nanoparticles is realized, and the problem of extremely low permeation efficiency of proteins in the intestinal epithelial cell layers is solved. Including but not limited to transferrin, neonatal Fc receptor protein, cell penetrating peptide, octaarginine, and the like.

The preparation method of the metal organic framework nano-particle for oral protein administration comprises the following steps: adding the micromolecule protein/polypeptide or the solution thereof into the acid-resistant metal organic framework water solution, and continuously stirring to ensure that the acid-resistant metal organic framework is coated with the micromolecule protein/polypeptide; and adding the targeting molecule or the solution thereof, continuously stirring to enable the targeting molecule to be adhered to the acid-resistant metal organic framework, and removing the small molecular protein/polypeptide or the targeting molecule which is not carried or adhered by centrifugation to obtain the metal organic framework nanoparticle for oral protein administration.

In some embodiments, the above-described method for preparing metal-organic framework nanoparticles for oral protein administration can be performed at about 4-about 35° C., about 10-about 25° C., or about 15-about 20° C.

In some embodiments, the metal organic framework nanoparticle for oral protein administration comprises an acid-resistant metal organic framework of UiO-68-NH₂. The small molecular protein/polypeptide can be insulin, and the targeting molecule can be transferrin. Wherein, in UiO-68-NH₂, the mass ratio of the insulin to the UiO-68-NH₂ can be about 1:1-about 1:6, about 1:1-about 1:5, or about 1:1-about 1:3, and in UiO-68-NH₂, the mass ratio of the transferrin to the UiO-68-NH₂is preferably about 1:0.1-about 1:1, about 1:0.3-about 1:1, or about 1:0.5-about 1:1.

The acid-resistant metal-organic framework nanoparticles (UiO-68-NH₂) can encapsulate sufficient insulin and decorate the exterior with targeting proteins (transferrin) to realize highly efficient oral insulin delivery. The UiO-68-NH₂ nanocarrier with proper pore size can achieve high insulin loading while protecting insulin from acid and enzymatic degradation. Through receptor-mediated transcellular pathway, the transferrin-coated nanoparticles may realize efficient transport across the intestinal epithelium and controlled insulin release under physiological conditions, leading to a remarkable hypoglycemic effect and a high oral bioavailability of about 29.6%. Thus, functional metal-organic framework nanoparticles can protect proteins from the gastric environment and overcome the intestinal barrier, thus providing the possibility for oral biomacromolecule delivery.

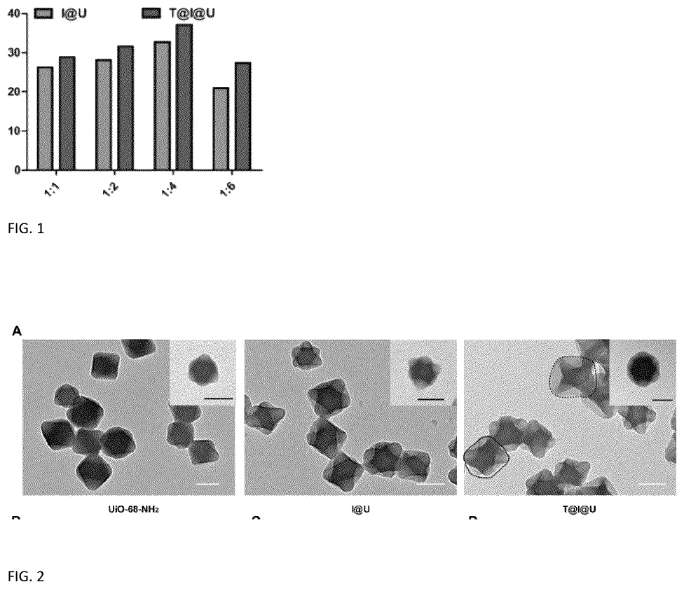

In some embodiments, utilizing acid-resistant nMOFs (e.g., UiO-68-NH₂) with exterior targeting protein (Tf) decoration, a nanosystem can be constructed with protein protection and intestinal transportation functions for the highly efficient oral INS administration. The selection of the UiO-68-NH₂ nanoparticles (NPs) is primarily based on their sufficient porosity, acid stability, and phosphate ions instability. The ligand molecule of UiO-68-NH₂ is longer than that of most MOFs, leading to a larger pore structure with a diameter of about 2.6 nm to load sufficient INS (about 2 nm in monomeric form) but exclude pepsin (about 6 nm). This steric hindrance effect between UiO-68-NH₂ and macromolecule proteases can protect INS from proteolysis in the GI tract. The acid stability and phosphate ions instability of UiO-68-NH₂ can protect INS as well in harsh acidic environments and realize controllable INS release under phosphate-rich physiological conditions. As for the targeting protein, Tf meets the critical requirements of the targeting agent for oral administration: (1) Intestinal epithelial cells express a large number of receptors for Tf to combine; (2) Tf has good stability in the GI tract as well due to the ability to partially resist the degradation of trypsin and chymotrypsin. Consequently, in this study, INS can be loaded into highly porous UiO-68-NH₂ NPs (denoted as I@U) and further coated the exterior side with Tf on (T@I@U), as illustrated in FIG. 1. The encapsulated INS within the UiO-68-NH₂ NPs can be protected from the gastric environment including harsh acid and protease, and exhibit controlled release in physiological conditions. Moreover, the Tf-coated acid-resistant MOFs oral delivery nanosystem can be rapidly absorbed by intestinal cells and escape from lysosomes for deeper penetration, which is adequate for rapid and efficient intestinal transportation and ultimately achieving a favorable therapeutic effect. In addition, the preparation of such a nanosystem, based on a single-step solvothermal reaction from readily available starting materials, is simple, low-cost, and easily scale-up. The targeting protein-coated acid-resistant nMOFs can be utilized for oral INS administration, displaying the great potential of efficient oral protein delivery.

The preparation method for some embodiments comprises the following steps:
(a) dissolving insulin powder in dilute hydrochloric acid solution, slowly dropwise adding the insulin hydrochloric acid solution into UiO-68-NH₂ newly synthesized by solvothermal method under the condition of stirring at room temperature. In an aqueous solution and stirring is continued for about 0.1 to about 12 hours, about 1.0 to about 10 hours, or about 3 to about 8 hours to obtain insulin @metal organic framework nanoparticles (I @ U). (b) Subsequently, the transferrin powder is dissolved in the aqueous solution, and the transferrin solution is added to the solution obtained in (b) with stirring at room temperature and stirring is continued for about 0.1 to about 12 hours, about 1.0 to about 10 hours, or about 3 to about 8 hours to obtain transferrin @ insulin @ metal organic framework nanoparticles (T @ I @ U). The unencapsulated or unattached proteins were removed by centrifugation.

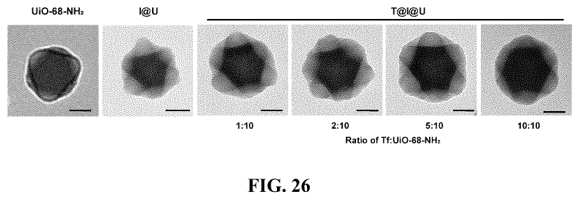

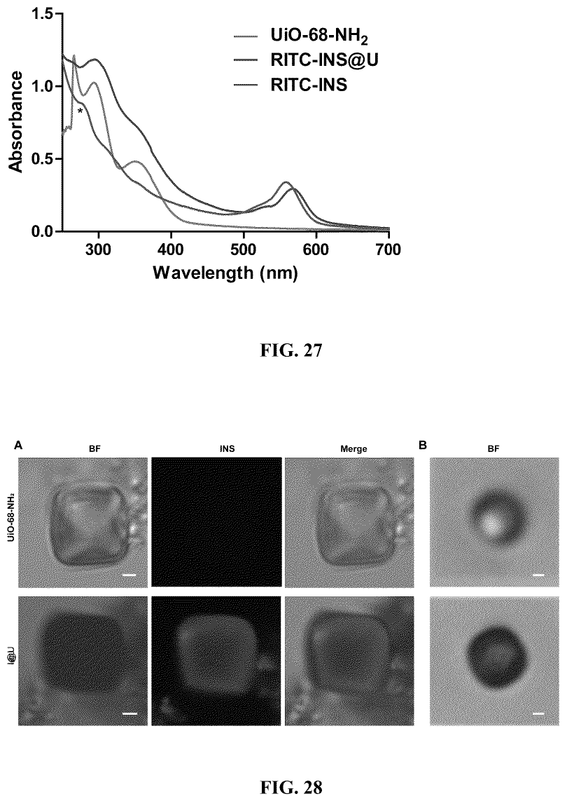

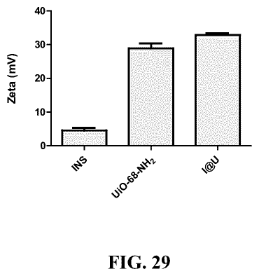

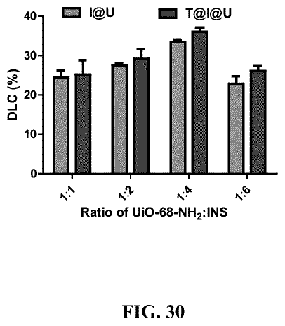

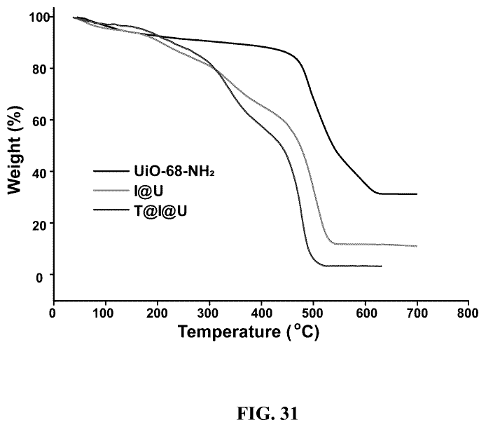

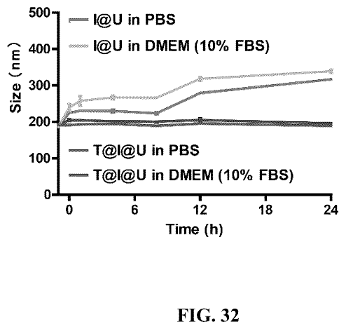

In some embodiments, preparation of nMOFs-based protein delivery nanosystems for UiO-68-NH2 NPs, I@U NPs, and T@I@U NPs can be as follows. UiO-68-NH2 NPs with uniform regular octahedron shapes are obtained through the facile one-step solvothermal method. The changes in morphology and particle size of NPs are negligible after loading the INS. The hydrodynamic diameters of UiO-68-NH₂ and I@U are 156.4±1.9 and 157.7±0.2 nm, respectively, and the angles of the regular octahedron are relatively sharp. When varying amounts of Tf are decorated on the surface via physical adhesion, the angles of NPs gradually become blunt, as depicted in FIG. 26. The morphology of NPs after Tf coating gradually changes from a regular octahedron to a sphere, and the size increases to 171.7 plus or minus (±) 3.4 nm. The zeta potentials of UiO-68-NH₂ NPs rose from +25 mV to +35 mV after loading with INS and nearly approached +40 mV after further decoration with Tf. See, e.g., FIG. 2. The successful immobilization of RITC-labeled INS in UiO-68-NH₂ NPs is confirmed by UV-vis spectroscopy, in which the maximum absorption of RITC slightly redshift from 558 nm of free RITC-INS solution to 567 nm of the encapsulated form, as depicted in FIG. 27. The confocal lasers scanning microscopy and optical images further indicate that RITC-labeled INS is distributed throughout the UiO-68-NH₂ crystals, as depicted in FIG. 28. Although not wanting to be bound by theory, owing to the positively charged nature of UiO-68-NH₂ NPs and INS at the acidic condition (pH=4) of the loading process, as depicted in FIG. 29), the INS encapsulation is mainly driven by hydrophobic interactions between the hydrophobic surface of UiO-68-NH₂ and INS instead of electrostatic interactions. Additionally, FITC-labeled Tf and RITC-labeled INS are used in preparing the dual-fluorescence-labeled nMOFs to prove the formation and the structural integrity of T@I@U. FITC and RITC are commonly used as fluorescence resonance energy transfer (FRET) fluorescence pairs. FRET phenomenon can occur when the distance between FITC and RITC is less than 10 nm. The fluorescence spectra can be exhibited where a strong FRET signal can be observed. The emission intensity of FITC-Tf decreases sharply at 520 nm, while that of RITC-INS significantly increases at 585 nm, clarifying the co-existence and spatial proximity of INS and Tf in T@I@U NPs. The powder X-Ray diffraction (PXRD) patterns of both I@U and T@I@U NPs are essentially the same as that of UiO-68-NH2 NPs and the calculated pattern from UiO-68-NH₂ single crystals, which demonstrates that UiO-68-NH₂ maintains its crystallinity throughout the encapsulation and decoration process. In addition, the energy dispersive spectroscopy (EDS) elemental mapping analyses are tested to confirm the existence of each component in T@I@U NPs (S for proteins, Fe for Tf, and Zr for UiO-68-NH₂). Elements of S, Zr, and Fe can be uniformly distributed on the NPs, and the range of S is significantly more extensive than that of Zr, which provides direct evidence for the successful fabrication of T@I@U. Macromolecules of INS can be loaded into UiO-68-NH₂ successfully with Tf uniformly decorated on the I@U NPs. The INS-loading behaviors of UiO-68-NH2 NPs are further explored using the bicinchoninic acid (BCA) method by simply mixing INS and UiO-68-NH₂ NPs at a mass ratio from about 1:1 to about 6:1, about 1:1-about 1:5, or about 1:1-about 1:3, as depicted in FIG. 30. The UiO-68-NH₂ NPs exhibit a high loading capacity (LC) of 33 wt. % at the feeding ratio of 4:1, higher than some of the INS nanocarriers, as depicted in FIG. 39. The mass loss is also observed to determine the LC of the UiO-68-NH₂-based nanosystem by thermogravimetric analysis (TGA), which is consistent with the results, as depicted in FIG. 31. The high loading capacity and facile exterior modification of UiO-68-NH₂ can be a suitable candidate for INS nanocarrier.

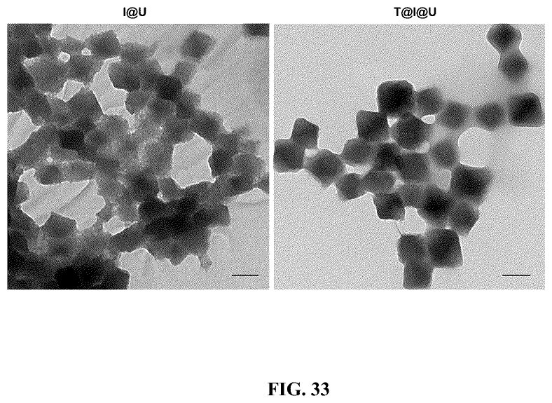

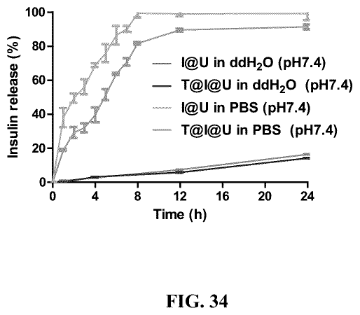

In some embodiments, the vitro stability and protein protection performance of Tf-coated nMOFs can evaluate the in vitro stability of the UiO-68-NH₂-based nanosystems. The changes in size and morphology can be monitored under various situations at different time intervals. No significant change in particle size is observed when different formulations are dispersed in a simulated gastric pH environment (dilute HCl, pH equals (=) 2) for 14 days, owing to the strong acid-proof stability of the UiO-68-NH₂. In contrast, the particle size of I@U increases significantly under the physiological condition because the UiO-68-NH₂ begins to clump together and disintegrate, see for example, FIGS. 33-34. Interestingly, after being coated with Tf, the deconstruction is greatly retarded, demonstrating that Tf decoration endows the nanosystem with slow-release performance in physiological conditions.

The ability of UiO-68-NH₂ for protecting INS is explored against protease degradation. As shown in FIG. 4, free INS is almost completely degraded after only 1 hour (h) in the trypsin-containing HBSS buffer, while about 80% of active INS remains in the I@U group. The result demonstrates that INS is protected from degradation due to a certain steric hindrance between INS and the protease after being encapsulated in UiO-68-NH₂. In addition, the residual amount of INS is higher than 50% after 2 h due to the protective effect of Tf, as mentioned above. Altogether, the protection effect of Tf-coated acid-resistant nMOFs provides a favorable prerequisite for the long-term circulation of nanosystems in vivo.

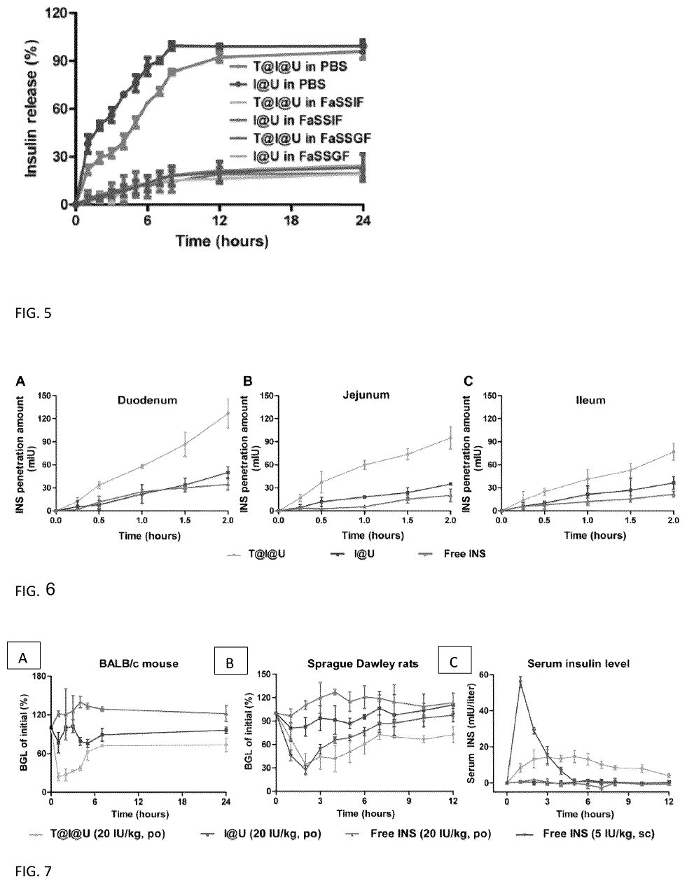

Sustained and controlled INS release kinetics of T@I@U can be undertaken with an effective oral delivery nanocarrier. The nanocarrier UiO-68-NH2 has proven its adequate protection for INS against severe conditions. Meanwhile, the steady release of INS from the acid-resistant nMOFs in physiological conditions is also demanded. The release profiles of INS from I@U and T@I@U under simulated gastrointestinal environment (FaSSGF for simulated gastric fluid and FaSSIF without phosphate for simulated intestine fluid) and simulated physiological conditions (PBS, pH=7.4) are shown in FIG. 5. Under the FaSSGF, FaSSIF, and ddH₂O environment, as depicted in FIG. 34, less than 20% of INS from I@U and T@I@U are released in 24 h. While under the PBS condition, INS is slowly released from UiO-68-NH₂, and the cumulative released INS from I@U and T@I@U reaches 99.1% and 90.3% at 12 h, respectively. Continuous release of INS under different environments is also evaluated to simulate the status of orally taken drugs. Barely any INS is released from neither I@U nor T@I@U under acidic to neutral pH conditions in the stomach and intestine. But after being incubated in a neutral PBS environment, I@U and T@I@U starts to deconstruct and release INS. The INS from I@U is almost completely released within 10 h in PBS, while the percent of released INS from T@I@U is only about 80%. Consistent with the stability result, Tf slightly hinders the disintegration of UiO-68-NH₂ and slows the release rate of INS. The above results indicate that the release of INS from Tf-coated nMOFs has considerable environmental sensitivity, which is specifically triggered by phosphate in PBS rather than pH. In general, T@I@U exhibits sustained and controlled release kinetics.

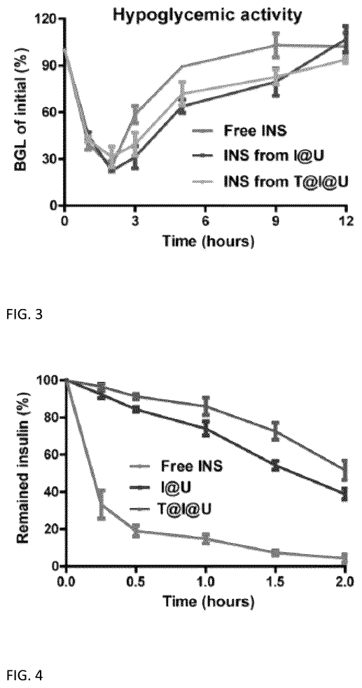

To further verify that the activity of INS is not destroyed, the bioactivity of INS after the drug release process by injecting free INS and INS released from PBS-treated I@U and T@I@U into diabetic mice subcutaneously is examined. As depicted in FIG. 3, the released INS from I@U and T@I@U produces a significant hypoglycemic effect analogous to an equivalent dose of free INS solution, indicating the process of INS immobilization in UiO-68-NH₂ does not impair its activity, which is essential for the subsequent experiments.

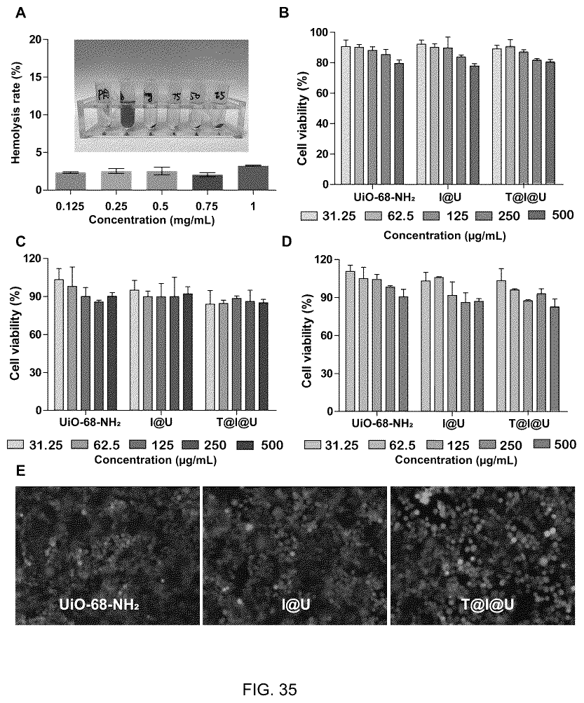

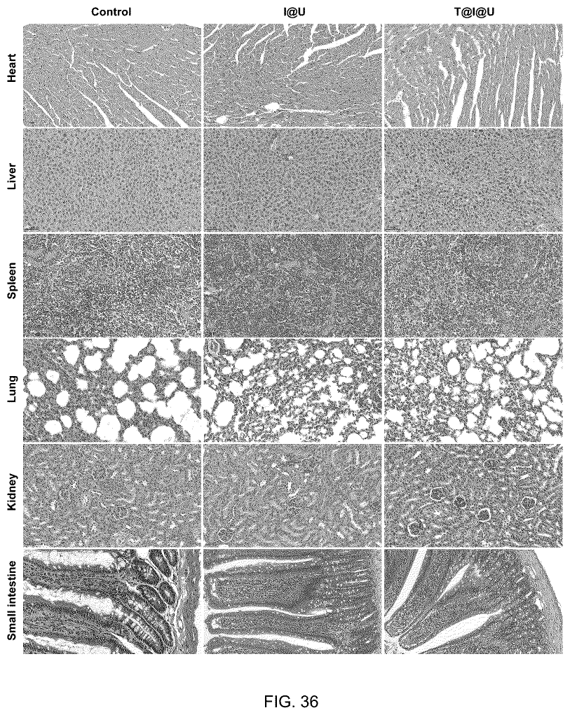

Biocompatibility and long-term safety analysis of the UiO-68-NH₂-based nanosystems are examined for biocompatibility through in vitro cell viability and live and dead imaging. As shown in FIG. 35, all samples exhibit negligible cytotoxicity (cell viability greater than 85%) on three different cell lines even when the concentration reaches 500 μg/mL, which is also confirmed through live and dead staining. To examine potential cell-membrane disruption, T@I@U is incubated with erythrocytes for 4 h, and there is no evidence of hemolysis. The hemolysis rate is 3.23% when the concentration reaches 1 mg/mL, verifying that T@I@U barely causes damage to cell membranes. Meanwhile, no significant reduction of trans epithelial electrical resistance (TEER) is observed when the cell monolayer is exposed to I@U and T@I@U, which confirms the integrity of the tight junctions. Inspired by the low toxicity of single-dose T@I@U, the potential long-term toxicity of the UiO-68-NH₂-based nanosystem is further examined via an extended in vivo experiment. As depicted in FIG. 36, the finger-like villi are observed in the small intestine tissue of mice, indicating the intact structure of the intestine after twice-daily T@I@U treatment for seven consecutive days. Also, no apparent structural damage occurs to the organs and small intestine tissue. The above results corroborate the good biocompatibility of the UiO-68-NH₂-based nanosystem.

The Tf-mediated enhanced cellular internalization and transepithelial activity of T@I@U can be similar to the structure and function of small intestinal epithelial cells. Human colon adenocarcinoma (Caco-2) cells are currently used to study the transportability of drugs across the intestinal barrier to predict drug absorption. This research utilizes Caco-2 cells as an in vitro model and determines whether Tf-coated nMOFs can traverse the intestinal epithelium by TfR-mediated endocytosis and successfully escape the lysosome for deeper penetration. The qualitative and quantitative uptake of the above-mentioned fluorescence-labeled UiO-68-NH₂-based NPs by Caco-2 cell monolayers can be determined. Because INS is a biological macromolecule with high polarity and strong hydrophilicity, INS has poor passive diffusion and transmembrane ability. Only a small amount of fluorescence is observed in the free INS and I@U groups, while the Tf-coated nMOFs exhibit a much stronger fluorescence signal compared with other groups. The results demonstrate a TfR-mediated better uptake effect, which is further confirmed by the deeper fluorescence distribution along the z-axis and the flow cytometry analysis. The quantitative cellular uptake level of T@I@U is approximately 2.25 times that of free INS and 1.5 times that of I@U groups. It is worth noting that TfR-mediated cell internalization is time-dependent, and the cell uptake level is as high as 53.2% in only 30 minutes (min), indicating the rapid and effective internalization of Tf-coated nMOFs.

As mentioned above, Tf-coated nanocarriers undergo transcytosis by binding to TfR on epithelial cell membranes. Therefore, to confirm the fate of Tf-coated nMOFs during the transport process, the distribution of dual-fluorescence-labeled nMOFs in Caco-2 cell monolayers is explored. After using T@I@U to incubate the cells for only 5 min, the green fluorescence of Tf is distributed around the cell, which indicates that the receptor rapidly binds to the cell membrane. After 30 min of incubation, the fluorescence location results demonstrate that Tf has quickly and effectively transferred the nMOFs into the cell monolayers. In addition, the cell monolayer pre-incubates with an excessive amount of free Tf as the competitive inhibitor. The free Tf preferentially binds with the TfR on the apical side while the cellular uptake level of T@I@U drops significantly to 62%. The above results demonstrate that the uptake of T@I@U mainly occurs through TfR-mediated active endocytosis.

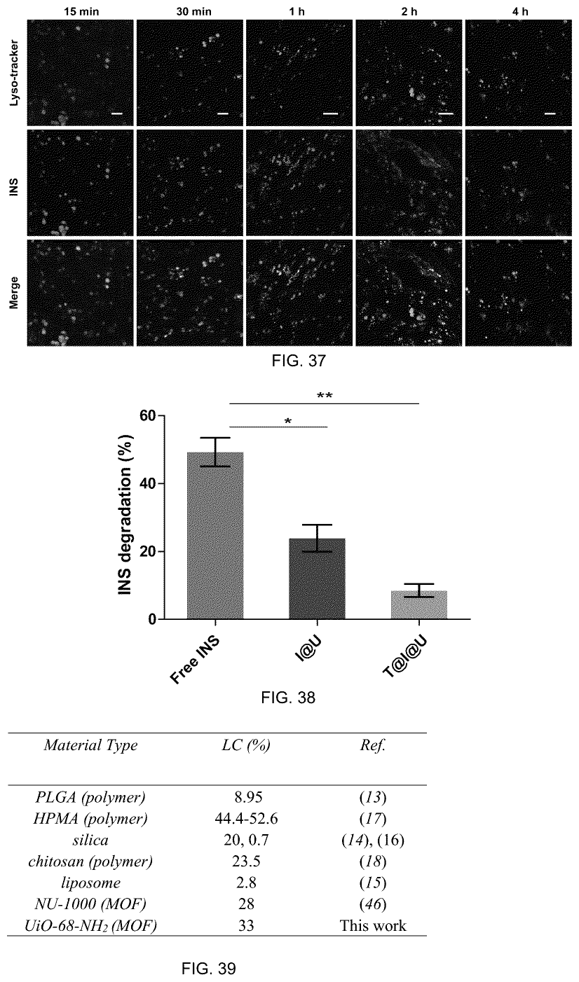

After endocytosis, the nMOFs can be transported to the lysosome for digestion. Although, as demonstrated above, nMOFs can partially protect INS from being degraded by proteases, INS may gradually become inactive if failing to escape the lysosome. Therefore, the behavior of Tf-coated nMOFs can be explored when interacting with lysosomes via CLSM. Mechanically, the Tf-coated acid-resistant nMOFs have a strong positive charge and a unique membrane switching effect through electrostatic interaction with the lysosome membrane, leading to lysosome escape. As shown in FIG. 37, after just 15 min of incubation, almost all the fluorescence signal of T@I@U is trapped within the lysosome, confirming that nMOFs can be quickly internalized through the TfR-mediated pathway. As the incubation time prolongs to 2 h, the colocalization of the T@I@U and lysosome is reduced sharply, further indicating an effective escape from the inner lysosome compartment to avoid INS degradation. The co-localization rate slightly increases at 4 h of incubation, implying that the escaped nMOFs might be re-internalized by the cells without removing the incubation media. The INS degradation rate of different formulations is detected in the Caco-2 cell monolayers. As shown in FIG. 38, about 50% of ingested INS in the free INS group is degraded after 2 h of incubation, while the I@U and T@I@U groups significantly inhibit the degradation rate of INS. Only about 25% and less than 10% of INS are degraded, attributed to the protection of the UiO-68-NH₂ and the ability of T@I@U to escape the lysosome.

At length, the basolateral membrane is the last barrier hindering the transport of INS into the bloodstream circulation. Hence, the transcellular transportation through the basolateral side of different preparations is evaluated. Firstly, the transepithelial permeability of INS from different formulations across the Caco-2 cell monolayer is analyzed. In just 5 min, the INS transport through the cell monolayer in the T@I@U group exceeds 20%. The cumulative penetration INS reaches 80% after 2 h, far exceeding the free INS and I@U groups. Meanwhile, the apparent permeability coefficient (Papp) is an essential parameter for investigating the permeability of drugs. After careful calculation, the Papp of T@I@U is 16.8×10-7 cm/s, 8.05- and 2.98-fold higher than that of free INS and I@U. The apparent changes of Papp indicates that Tf has significantly increased the penetration of INS through the cell monolayer, indicating that the nanosystem has entered into the cell in large quantities and successfully exocytosis from the basal side. Moreover, the transcellular transport of T@I@U is further evaluated by protocol. The INS signal of I@U and free INS groups have limited transportation among different batches of cells. In contrast, the fluorescence signal from T@I@U is high on coverslips (ii) and (iii). This phenomenon demonstrates that the INS took up on coverslip (i) is exocytosis into the medium and subsequently internalized by the cells on coverslips (ii) and (iii), which is conducive to drug penetration through the intestine. Overall, the apical endocytosis markedly increases via the TfR-mediated uptake pathway, and the protection of MOF reduces the degradation of INS in the lysosome. Hence, the permeability of INS across Caco-2 cell monolayers is significantly improved upon Tf-coated acid-resistant MOFs.

Penetration efficiency of T@I@U in the isolated small intestine is improved as the intestinal epithelium layer has been considered the most challenging barrier to the oral absorption of nanocarriers. The ability of T@I@U to transport across small intestinal ex vivo after the Tf-coated nMOFs is found to be effective at the cellular level in previous in vitro experiments. The transport of free INS, I@U, and T @I@U across the intestinal epithelium in the duodenum, jejunum, and ileum loops is compared. As shown in FIG. 6, the INS permeation amounts of different preparations are all time-dependent, especially the T@I@U group. Compared with free INS and I@U NPs groups, the T@I@U exhibits the highest accumulative amounts of INS permeated through each small intestinal section. At 2 h, the infiltration amount of INS from the T@I@U group in the duodenum is approximately 127 mIU. Moreover, a gradual decrease in INS penetration from the duodenum to the jejunum to the ileum occurs as well, probably due to a gradual reduction of TfR and a gradual increase of mucus throughout the small intestine. The above results illustrate that the absorption of T@I@U in the small intestine is dependent on the Tf-targeted pathway, which corresponds with the results obtained in cellular studies.

The in vivo biodistribution, intestinal absorption and distribution of INS are further determined by Maestro In Vivo Imaging System and CLSM. The in vivo images can show the fluorescence signal and intensity distribution of RITC-INS from three different formulations within 24 h. After 4 h of post-administration, a strong fluorescence signal is observed in the mice treated with T@I@U, which is greater than the free INS and I@U groups. The strong fluorescence of the mice treated with T@I@U reaches the maximum at 8 h, and this phenomenon perseveres for 12 h, which provides solid evidence for the long-term effect of the T@I@U NPs.

The biodistribution of INS fluorescence is also observed in intestines and major organs after 4 h of administration. Notably, strong fluorescence signals of INS released from T@I@U are observed in the liver, indicating that INS might initially circulate through the portal veins to the liver, consistent with the endogenous INS secretion behavior. Free INS does not show any fluorescence signal in major organs, owing to the rapid degradation of proteins in the GI tract under extreme pH conditions and protease environment. Despite the fact that a small amount of INS from I@U in the liver is observed, the fluorescence signal of INS is fragile compared with the Tf-coated nMOFs group, representing the inefficient absorption of INS through the intestine without Tf decoration.

To further verify the potential pathway for the T@I@U to cross the epithelial layer, the intestine epithelial tissues are sectioned and processed for confocal imaging after 2 h of administration. The fluorescence signal located inside the epithelial cells confirms that INS from T@I@U is transported into the intestinal villi successfully, while barely any fluorescence has been found in the free INS and I@U group. The above results indicate that only the Tf-coated acid-resistant MOFs can significantly improve the permeability of INS in the intestine and realize INS transportation orally.

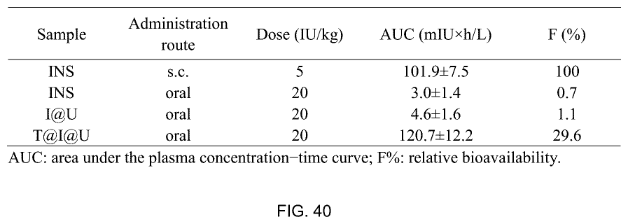

In vivo pharmacodynamics (PD) and pharmacokinetics (PK) studies investigate the hyperglycemic response and pharmacokinetics parameters of the INS-loaded nMOFs. The INS-loaded nMOFs (20 IU kg⁻¹) and free INS (20 IU kg⁻¹) are administered alloxan-induced diabetic rats and mice by gavage, and are subcutaneously injected the free INS (5 IUkg⁻¹) to alloxan-induced diabetic rats. The blood glucose and serum INS concentrations are monitored using a glucometer and bovine INS ELISA assay, respectively. As shown in FIGS. 7A and 7B, the oral administration of free INS and I@U fails to reduce the blood glucose level, while free INS (s.c.) group and T @I@U intragastric administration group generate a strong hypoglycemic response in contrast. Free INS (s.c.) group causes a sharp decrease of blood glucose levels (BGL) to about 28% of the initial level at 2 h, and the hypoglycemic effect subsequently wears off 4 h later. Compared with the free INS (s.c.) group, the T@I@U quickly reduces BGL to around 30% of the initial level in the first 2 h and led to a prolonged reduction in BGL during 8 h. Based on the glucose-decreasing profile, the relative pharmacological activity of T@I@U is calculated to be 31.9%. The corresponding serum INS profiles of rats treated with different formulations are depicted as the area under the curve (AUC₀₋₁₂ h) in FIG. 7C, representing a consistent result with pharmacodynamics. The maximum serum INS level of free INS (s.c.) appears within 1 h and returns to a minimum in only 4 h. Compared to the free INS group, the T@I@U group maintains a relatively high and stable serum INS level for up to 10 hours. The above experimental results indicate that Tf-coated nMOFs can prolong the INS efficacy in vivo and reduce the transient hypoglycemia side effects caused by the free INS injection. The pharmacokinetic parameters calculate from peripheral serum INS are shown in FIG. 40. The AUC of T@I@U is 120.7 mIU×h/L with a relative bioavailability of 29.6%, which is 42.3 and 26.9-fold higher than oral INS solution (0.7%) and the I@U group (1.1%), respectively.

In some embodiments, a simple, efficient, and sustained-release oral insulin delivery system based on folic acid (FA)-conjugated acid-resistant metal-organic framework (MOF) nanoparticles with high drug loading capacity can be used. The FA-conjugated MOF (PCN-777) not only selectively enhances its intestinal transportation efficiency in diabetic animals through up-regulated intestinal FA transporter-mediated endocytosis, but also maintains long-term basal insulin release kinetics by delaying the disintegration of PCN-777 in the phosphate-rich environment. In diabetic animals, the PCN-777-based oral insulin dosage form exhibits a prolonged hypoglycemic effect for up to 48 hours and a high bioavailability of 35.5%, representing a potential once-every-two-day formulation. The extended drug duration and enhanced efficacy of MOF-based oral protein/peptide delivery nanosystems offers possible translational opportunities for long-term glycemic management.

Proteins and peptides have emerged as ideal drugs for the treatment of various chronic and acute diseases due to their high efficiency, high affinity, high selectivity, and low toxicity. Nevertheless, most approved therapeutic proteins and peptides have short half-lives and are administrated exclusively via continuous subcutaneous injection, leading to great pain and inconvenience. For instance, the duration of regular insulin (INS) action is generally between 4-8 hours, which means that people with severe diabetics must take 2-4 INS injections per day to manage blood glucose levels properly. Unfortunately, it is reported that 45-60% of diabetic patients intentionally skip one or more doses due to the fear associated with serial injections, resulting in suboptimal glycemic control. Therefore, it is of great significance to develop a non-invasive INS delivery system capable of maintaining long-term basal INS release kinetics. Oral administration of protein/peptide is considered a life-altering solution for the treatment of chronic diseases, with the advantages of convenience, safety, and good patient compliance. In 2019, an oral semaglutide tablet (sold under the trade designation Rybelsus®), became the first oral protein/peptide drug approved to launch by the FDA around the world, and brought exciting news to the field of oral protein delivery research, which is of profound significance. Although Rybelsus® has a good effect on hypoglycemic and weight loss, the clinical application is still limited by the low bioavailability of about 1.4%. The major difficulty in developing oral protein delivery systems is the multiple physiological barriers found in the gastrointestinal tract, including ultra-acidic pH, proteolytic enzymes, mucus layer, and intestinal epithelium. To improve the oral bioavailability of protein and peptide drugs as much as possible, researchers have screened numerous nanocarriers that can overcome the above physiological barriers, including liposomes, polymeric nanocarriers, silicon nanoparticles, etc.

Among various nanocarriers, nanoscale metal-organic frameworks (nMOFs) are a relatively new class of porous nanomaterials that combine the advantages of high drug-loading capacity, excellent biocompatibility and biodegradability, controllable drug release, and easy modification. Therefore, nMOFs have received extensive attention in drug delivery in recent years. For example, a pH-triggered self-disintegrating capsule based on a zwitterionic hydrogel-coated MOF for oral peptide delivery can significantly increase endogenous INS secretion. Additionally, a transferrin-coated acid-resistant Zr-MOFs oral INS delivery system with efficacious protein protection, intestinal transportation, and controlled release properties has been developed. Nevertheless, the short duration of drug action caused by MOF disintegration has greatly hindered the further clinical application of MOF-based oral INS delivery nanosystems.

The disintegration of Zr-MOFs in vivo is specifically triggered by the phosphate environment, primarily attributable to the competition for the excessive Zr cluster coordination sites between the phosphate group and the carboxylate group of the linker. From this, although not wanting to be bound by theory, the use of functional molecules with a carboxylic terminus to occupy the redundant coordination sites on the Zr₆ cluster can impede the attack of phosphate, thereby achieving the desired effect of sustained release. Folic acid (FA), a carboxylate-containing compound, can easily bind to the vacant sites on Zr₆ clusters, and in turn can generate a sustained-release effect under physiological conditions through steric hindrance. More significantly, a unique intestinal pathological characteristic in diabetic animals is reported where the expression of proton-coupled folate transporter (PCFT) in intestinal epithelial cells is up-regulated at both mRNA and protein levels. In that case, it is possible that FA-conjugated MOF can gain the capability to favorably alter the transcellular selection of material (i.e. a conjugated drug delivery platform) through the upregulated transporters mediating endocytosis and thereby facilitating oral INS delivery, particularly in diabetic animals. Therefore, the combination of FA and MOF is not only expected to improve the intestinal penetration efficiency of MOF nanoplatforms for oral delivery but also may slow down the disintegration of MOF in a phosphate environment.

Herein, a design of an oral sustained INS delivery platform based on functional molecules (FA) conjugated mesoporous acid-resistant MOF (PCN-777) is made. The selection of PCN-777 is primarily based on the fact that it possesses the largest cage size of 3.8 nm among the reported Zr-MOFs, allowing a considerable amount of INS to be encapsulated. PCN-777 also displays a high level of acid-aqueous solution stability despite its high porosity, which makes PCN-777 a suitable candidate for INS oral delivery. Furthermore, the coordination sites of the Zr₆ cluster in PCN-777 are partially occupied by six carboxylate organic linkers, leaving abundant positions to be occupied by terminal OH and H₂O groups, which can be facilely substituted by FA under mild conditions so as to achieve sustained release and enhanced oral administration of INS. Overall, the FA-conjugated PCN-777 proposed as an oral INS delivery platform also possesses the advantages that have been previously reported for MOF nanosystems, such as protein protection, controlled release, effective intestinal penetration, and, superiority of sustained release coupled with a selectivity hypoglycemic effect (only in diabetic animals). Thus, the concept that MOF can achieve long-term protein delivery after proper modification, taking MOF-based drug delivery system one step further toward clinical applications.

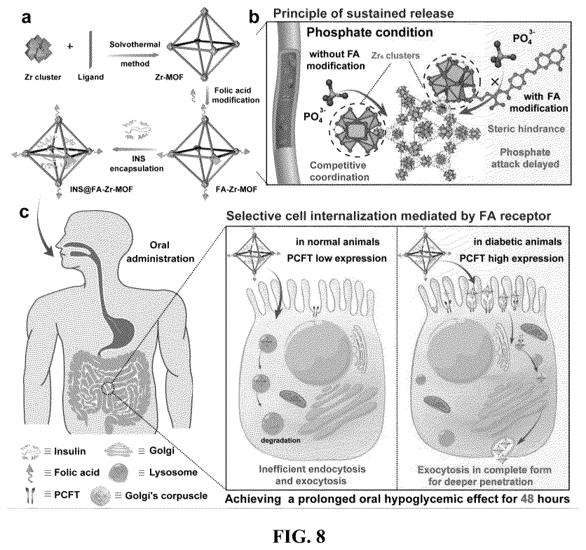

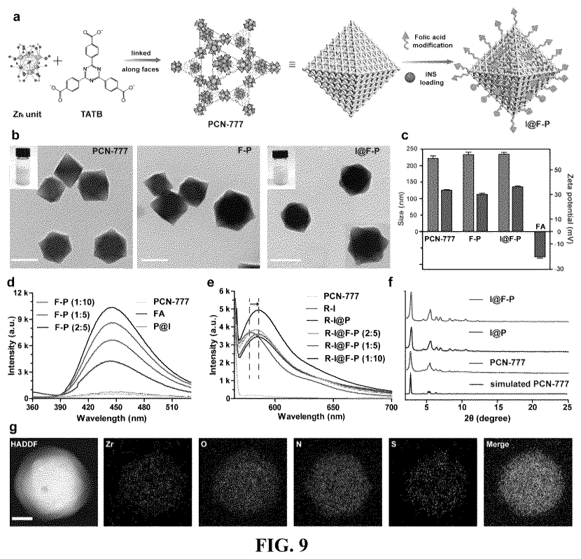

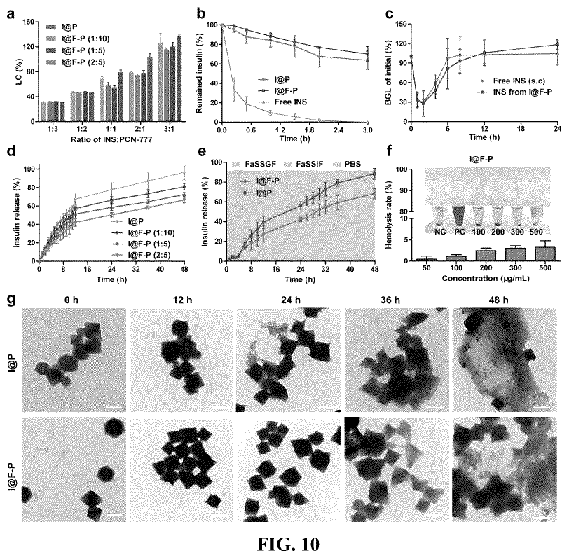

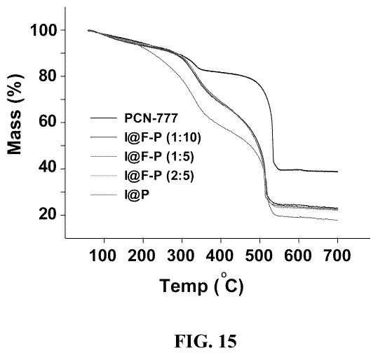

The preparation procedures of PCN-777, F-P, I@P, and I@F-P are depicted in FIG. 8a. The particle size of PCN-777 is commonly above 500 nm, which is not conducive to crossing the intestinal barrier. Therefore, the PCN-777 nanoparticles (NPs) are first synthesized with a particle size distribution of about 220 nm through the optimized solvothermal method. The TEM images and hydrodynamic diameters reveal that the PCN-777-based nanosystem is composed of homogeneous regular octahedrons, as depicted in FIGS. 8b and 8c. After FA modification and INS encapsulation, the color of the NPs turns yellow, the surface changes from smooth to rough, and the particle size slightly increases. The PCN-777-based nanosystem also exhibits non-negligible zeta potential changes during the consecutive encapsulation process; first, decreasing from 33 mV to 30 mV due to the negative charge nature of FA, and subsequently increasing to 36 mV after INS loading, as depicted in FIG. 9c. The fluorescence spectroscopy and UV-Vis spectroscopy measurements are performed to further confirm the modification and immobilization of FA and RITC-labeled INS in PCN-777. The fluorescence spectrum of FA-PCN-777 (F-P) shows the characteristic absorption peak of FA at about 450 nm, as depicted in FIG. 9d, illustrating the successful modification of FA. Likewise, the R-I characteristic absorption peak at about 560 nm of RITC-INS@FA-PCN-777 (R-I@F-P) in the fluorescence and UV-Vis spectra provides the expected evidence of INS encapsulation, as depicted in FIG. 9e. Notably, the absorption peaks of R-I in the R-I@P and R-I@F-P groups are red-shifted by about 4-5 nm. This is attributed to the aggregation of R-I molecules in the PCN-777 NPs, providing further evidence that insulin is entrapped in the PCN-777 NPs in large quantities rather than dispersed on the surface. The powder X-ray diffraction (PXRD) patterns of I@P and I@F-P NPs are essentially identical to that of PCN-777 NPs, as depicted in FIG. 9f, indicating that PCN-777 NPs maintains good crystallinity during the process of FA modification and INS encapsulation. The energy dispersive spectroscopy (EDS) elemental mapping analyses, as depicted in FIG. 9g, shows that the elements of Zr, S, N, and O are uniformly distributed on the NPs, proving the successful synthesis of nanosized PCN-777 and existence of INS. A first synthesized a FA-modified PCN-777 nanosystem has the appropriate size and uniform shape for INS oral delivery. In addition, the degree of FA functionalization has a crucial impact on the particle size, sustained release effect, and cellular uptake of NPs. Therefore, a series of FA-modified PCN-777 can be prepared in different proportions. The loaded amount of FA on the surface of PCN-777 NPs can be measured through the standard curves with a good linear relationship. As the feeding ratio of FA to PCN-777 increases from 1:10 to 4:10, or about 2:10 to about 3:10, the amount of surface-installed FA raises from 41 mg FA/g MOF to 218 mg FA/g MOF. Nevertheless, F-P NPs with different FA densities show a negligible effect on the limit of INS that could be encapsulated per gram of PCN-777, as depicted in FIG. 10a. This is likely due to the fact that FA mainly modifies PCN-777 by coordinating to Zr₆ clusters on the surface without occupying the pores. PCN-777 shows a considerable loading capacity (LC) of INS with a maximum of up to 121.5 wt. % when the INS to PCN-777 feeding ratio is 3:1. Consequently, the three components in the I@F-P NPs exists in the weight ratio of 50:41:9 (INS:PCN-777:FA), in which the loading content of INS is as high as about 50 wt. %. The thermogravimetric analysis (TGA) also verified the extraordinary loading capacity of INS by PCN-777 NPs, as depicted in FIG. 15.

In addition to the demand for high drug loading, clinical applications put forward higher requirements for the uniformity and stability of nanomedicine. Therefore, the stability of the PCN-777-based nanosystem under prolonged incubation time in solutions of ddH₂O or serum-containing medium by DLS analysis is explored. No significant change in particle size is observed in ddH₂O, proving that the PCN-777-based nanosystem has good water stability. Although the particle size of I@F-P NPs slightly increases up to 300 nm in the complete medium, there is no significant difference between the nanoparticle sizes under different incubation conditions. These results prove that the PCN-777-based nanosystem would not aggregate in large quantities in the simulated in vivo environment and lead to potential dangers such as blood vessel blockage. The PCN-777 has the ability to protect INS from protease degradation similar to UiO-68-NH2. As illustrated in FIG. 10b, about 70% of the active INS remained in the F-P group after 3 hours of incubation with trypsin buffer, exhibiting a more profound protein protect ability than UiO-68-NH2. This may be related to the degree to which INS is densely packed in the interior of the PCN-777-based nanosystem. To further verify the retention of biological activity of INS during the embedding, storage, and release process, the I@F-P NPs with PBS two months after the synthesis is disrupted and then injected the released INS subcutaneously into diabetic rats. As depicted in FIG. 10c, the INS released by I@F-P had a significant hypoglycemic effect on diabetic rats, similar to the equivalent dose of free INS. Thus, demonstrating that the FA-modified PCN-777 nanosystem could effectively prevent drug performance loss during INS embedding, long-term storage, and release process. Overall, the PCN-777-based delivery system is equipped with high drug loading capacity, high stability, and good protein protection, which facilitates the storage, transportation, and subsequent efficient oral delivery of protein drugs.

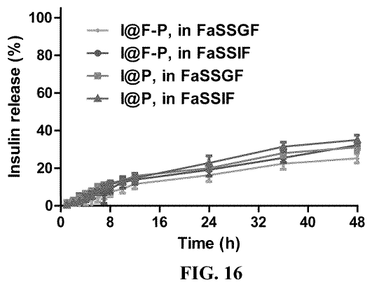

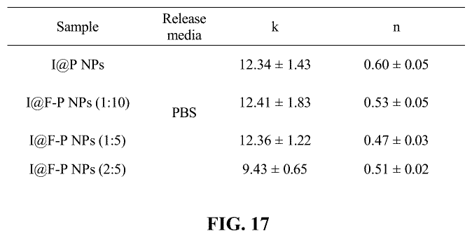

The sustained and controlled INS release kinetics of I@-P is analyzed. The stability and release profile of the PCN-777-based nanosystem is tested in different physiological conditions, as depicted in FIG. 10d and FIG. 16. The I@P group is released less than 35% of INS in 48 hours in both the simulated gastric fluid (FaSSGF) and simulated intestinal fluid (FaSSIF) environment, while the I@P continuously and slowly releases up to 93% of INS within 48 hours in phosphate-buffered saline (PBS). To simulate the dissolution and release of orally administered drugs at multiple levels in different media of the GIT, the continuous release of I@P NPs and I@F-P NPs in FaSSGF, FaSSIF, and PBS environments is further explored. Consistent with the previous results, I@P and I@F-P barely releases INS under acidic conditions. But after being incubated in a neutral PBS environment, INS from I@P and I@F-P NPs releases slowly, as depicted in FIG. 10e. These results demonstrate that PCN-777 is stable with little release INS in the gastrointestinal tract and disintegrates with a sustained rate under phosphate-containing conditions. In the above release experiments, the cumulative release of INS at 48 hours from I@F-P is remarkably lower than that of the I@P group and can be attributed to the higher PBS stability of FA-modified MOF. The release profiles of I@F-P with different FA/PCN-777 feeding ratios under phosphate-containing conditions can be explored. When the feeding ratio of FA to PCN-777 is increased to 2:5, the cumulative INS release of I@F-P decreased to 67% within 48 hours, as shown in FIG. 10d. The phosphate-triggered release is significantly mitigated by increasing amounts of FA modification. This phenomenon may be attributed to the steric hindrance between FA and PCN-777 which remarkably hinders the attack of phosphate on Zr₆ clusters, and thereby creates a quite specific process leading to the delay in the decomposition time of PCN-777. In addition, the release characteristic index n value can be calculated according to the Higuchi and Ritger-Peppas models. The n values of all nanosystems (I@P and I@F-P with different modification densities of FA) are greater than 0.45, as depicted in FIG. 17, which proves that the insulin release mechanism of PCN-777-based nanosystems is non-Fick diffusion (anomalous transport) due to the combined effect of drug diffusion and matrix erosion. Subsequently, to intuitively reflect the protective effect of FA on Zr-MOF, the morphological changes of I@P and I@F-P after incubation with PBS for different time intervals are further monitored. As shown in FIG. 10g, the I@F-P NPs becomes rough and starts to decompose slowly, yet the regular octahedral morphology is well maintained up to 36 hours. The absence of FA modification results in I@P NPs losing their basic morphology at 24 hours and completely cleaving at 48 hours. This result is consistent with the release profiles, which validates that the application of FA on Zr-based MOF can maintain the long-term curative effect and meet the requirements of clinical medication.

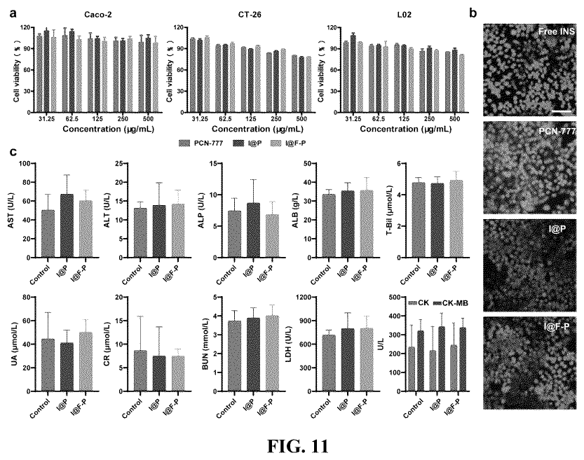

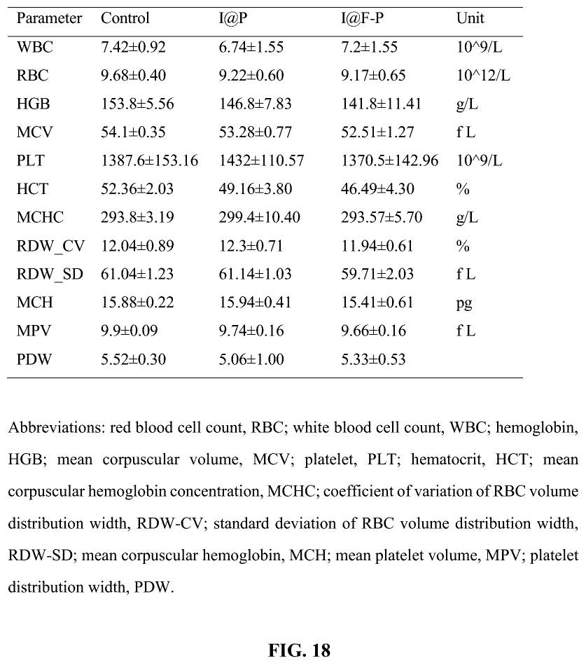

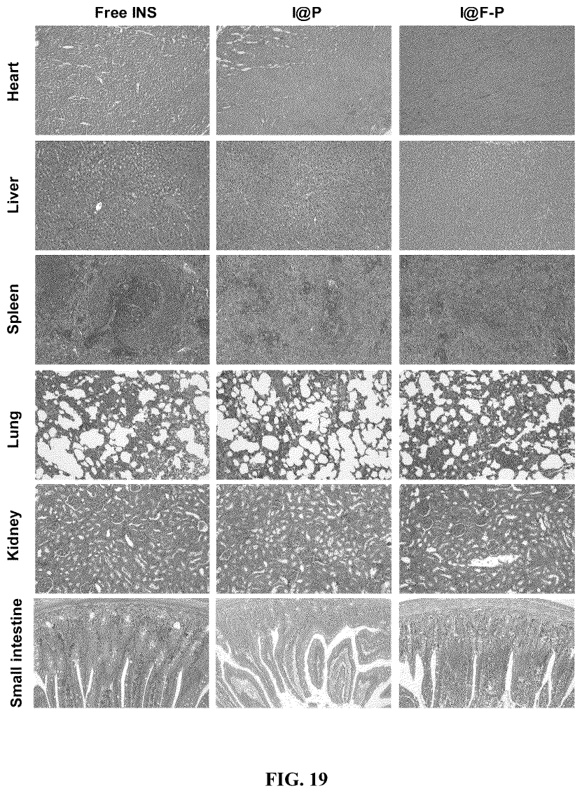

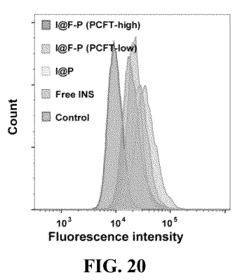

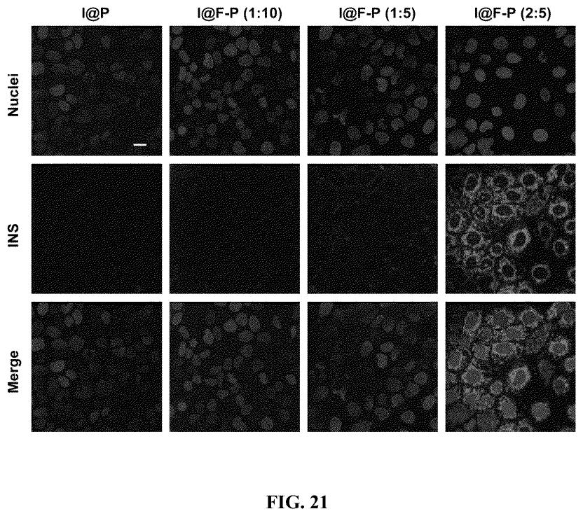

Biocompatibility and long-term safety analysis of I@F-P is conducted. Safety is the prerequisite for long-term medication in chronic diseases. The safety of PCN-777-based nanosystems is assessed on blood in vitro using an acute hemolysis assay. The hemolysis rate is 4.1% when administrated at a concentration of 500 μg/mL, as depicted in FIG. 10f), demonstrating that the PCN-777-based nanosystem has little damage to cell membranes and intracytoplasmic proteins. At the cellular level, MTT assays and live and dead cell imaging assays are performed. As shown in FIG. 11a, the PCN-777-based nanosystem shows low cytotoxicity on three different cell lines, and the cell viability is above 85% even when administered at a concentration of 500 μg/mL, which is consistent with the live and dead cell staining, as depicted in FIG. 11b. At the level of healthy and diabetic animal models, the potential long-term biosafety by exploring the changes in blood routine analysis is monitored, biochemical indicators and tissue morphology after oral administration of the PCN-777-based nanosystem every two days for two consecutive weeks. The results validate the biocompatibility of the material showing no significant difference in blood routine analysis and blood biochemical indicators between the PCN-777-based nanosystem group and the control group, as depicted in FIG. 11c and FIG. 18. Subsequently, the major organs and intestines are collected for H&E staining. As shown in FIG. 19, the intact structure of organs and small intestine tissues are observed and compared with the diabetic mice without drug administration. The above results corroborate the good biocompatibility of PCN-777-based nanosystem without significant side effects at the tested dose.

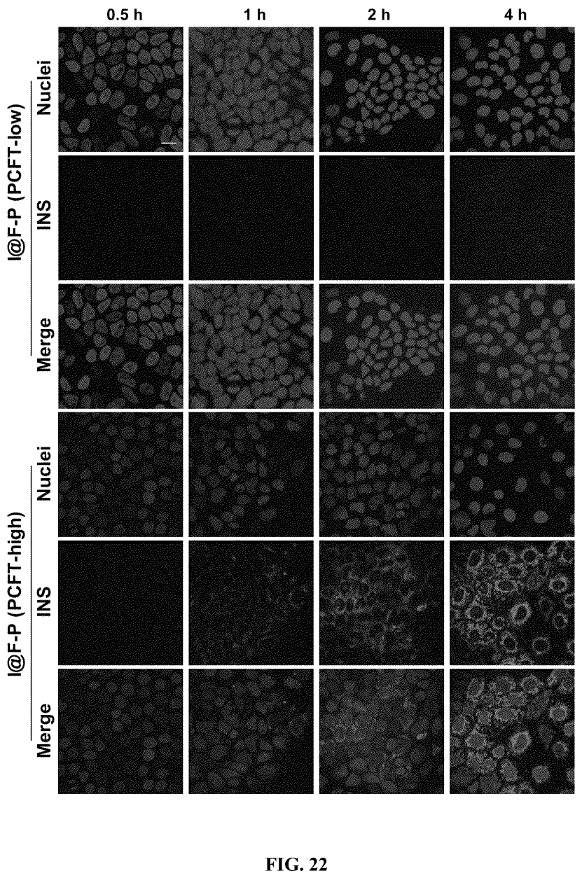

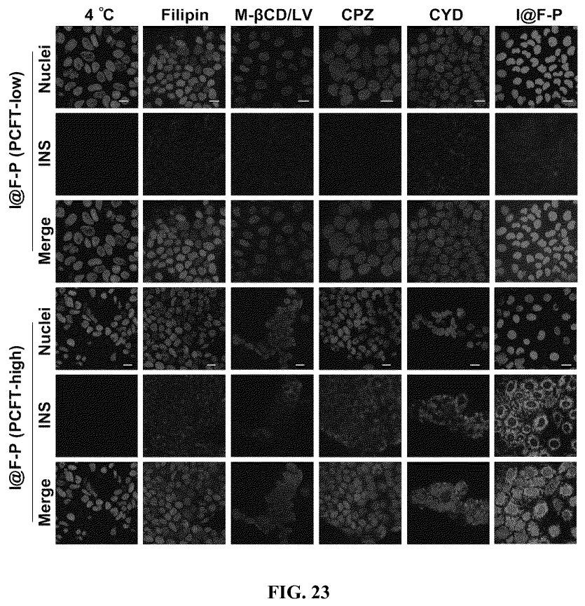

PCFT-mediated endocytosis of I@F-P NPs is studied. Human colon adenocarcinoma (Caco-2) cell lines with high expression of PCFT serve as the primary drug transportation cell model to predict the efficiency of I@F-P NPs intestinal absorption. Caco-2 cells with suppressed PCFT expression by myricetin are set as the control group to explore the impact on PCFT expression levels by the endocytosis of I@F-P NPs. The qualitative and quantitative uptake of PCN-777-based NPs in Caco-2 cell monolayers are visualized by CLSM and flow cytometry analysis, as depicted in FIG. 11a and FIGS. 21-22. The I@F-P NPs exhibit increased and time-dependent cellular internalization compared to other groups. In addition, the transportation depth of I@F-P NPs in the Caco-2 cell monolayer is deeper and closer to the basal side as evident from the red fluorescence distribution of the z-axis, as depicted in FIGS. 11a and 11b. According to the quantitative results obtained by flow cytometry, the cell uptake of I@F-P NPs is 9.9-fold and 2.1-fold that of Free INS, and I@P NP groups, respectively. Notably, the internalization of I@F-P NPs in PCFT-expressing suppressed cells is comparable to that of the I@P NP group, which confirms that the cellular internalization of the I@P NP group is mediated by the PCFT pathway.

To further investigate the endocytosis mechanisms of the I@F-P NPs, different endocytosis inhibitors while co-incubating the cells with I@F-P NPs following the previously reported protocols. As shown in FIG. 11c, 11d, and FIG. 23, the cellular internalization of I@F-P NPs decreases dramatically at 4° C. in both high and low-expression cell lines, suggesting that the I@F-P NPs cellular uptake is energy-dependent. The use of filipin and methyl-β-cyclodextrin and lovastatin can inhibit caveolin-mediated endocytosis by binding to cell membrane cholesterol and non-selectively depleting cholesterol. The cellular internalization of I@F-P NPs is reduced to 55% upon the addition of filipin and by about 58% when co-incubated with methyl-β-cyclodextrin and lovastatin, demonstrating that the caveolin pathway plays a significant role in the cellular internalization of I@F-P NPs. The addition of chlorpromazine suppresses the clathrin-mediated endocytosis, and the absorption of I@F-P NPs is reduced to 76% by chlorpromazine. This suggests that the clathrin pathway is also partly involved in the cellular uptake of I@F-P NPs. The cellular uptake of I@F-P NPs is slightly reduced after adding cytochalasin D for the inhibition of micropinocytosis. The above results are consistent with previous reports that caveolae-mediated endocytosis dominates the cellular uptake of I@F-P NPs, which may be related to the upregulated folate transporter. The clathrin-mediated endocytosis and micropinocytosis are also partially involved in the entry process of I@F-P NPs, but to a lesser extent. In contrast, the cellular uptake of the low-expression group is mainly through the clathrin-mediated pathway, which may explain the greatly reduced uptake in the low-expression group after inhibiting the PCFT. The above results demonstrated that upregulated PCFT alters the transcellular pathway of I@F-P NPs from caveolin-dominated to clathrin-dominated endocytosis.

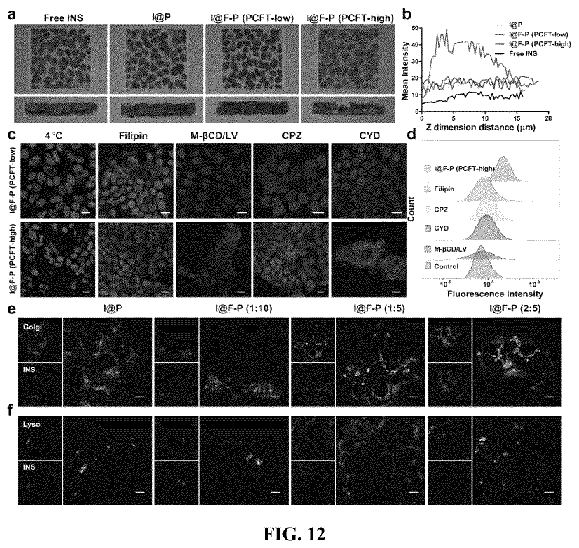

Intracellular localization experiments of PCN-777-based nanosystems to further confirm that upregulated PCFT mediates the transcellular transport. Previously, NPs are transported to lysosomes after clathrin-mediated endocytosis, leading to degradation, whereas NPs are transported to the Golgi apparatus after caveolin-mediated endocytosis and subsequently exocytosis in the intact form. As depicted in FIGS. 12e and 12f, almost all fluorescence signal of I@P NPs is co-localized with the lysosome rather than the Golgi. When the amounts of modified FA on the surface of the nanosystem increases, the co-localized organelles of I@F-P NPs gradually changes from the lysosome to Golgi. Thus, I@F-P NPs can be internalized through the PCFT-mediated pathway in Caco-2 cells with the upregulated PCFT, and further transported through the Golgi pathway.

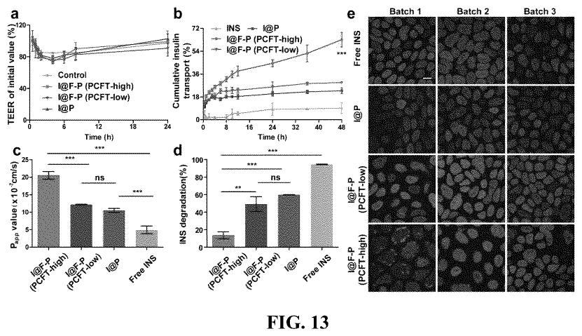

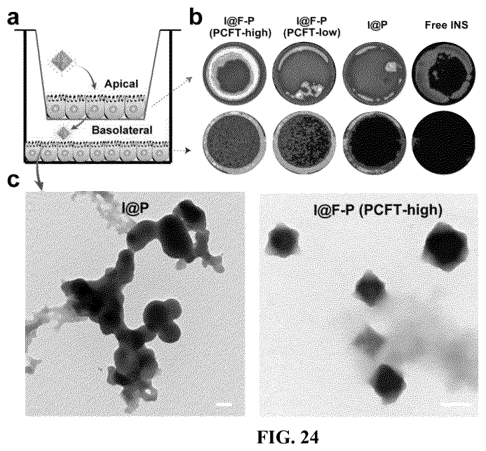

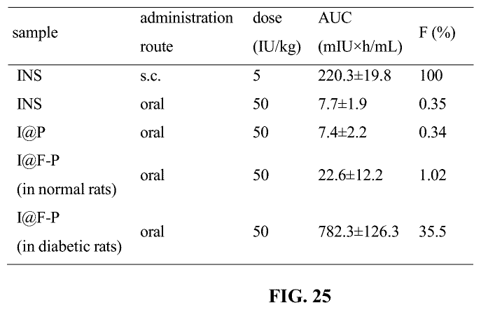

To verify whether I@P NPs can efficiently undergo exocytosis through the Golgi pathway, the transcellular permeability of PCN-777-based nanosystems in Caco-2 cell monolayers afterward is investigated. The transepithelial electrical resistance (TEER) values of Caco-2 cell monolayers during PCN-777-based nanosystems treatment is examined. No significant decrease in TEER is observed compared to the control group, confirming the integrity of the tight junctions, as depicted in FIG. 13a. The cumulative penetration INS of I@F-P in high PCFT expression cells reaches 62% at 48 h, which is 2.1-, 2.7- and 6.8-fold compared to I@F-P in low PCFT expression cells, I@P, and free INS, respectively, as shown in FIG. 13b. As shown in FIG. 24b, the fluorescence distribution of the INS preparations in the apical and basal cell layers is consistent with the above results. The apparent permeability coefficient (Papp) of I@F-P across high PCFT expression Caco-2 monolayers is 20.6×10⁻⁷ cm/s, as depicted in FIG. 13c. Thus, showing that the permeability of I@F-P is significantly improved in the high-expression group. Next, the culture medium on the basal side at 24 hours, is centrifugated and processed the precipitate for transmission electron microscopy. It can be seen in the corresponding figure, as depicted FIG. 24c, that in the high PCFT expression group, the I@F-P NPs maintain their complete morphological structure, while in the low expression group, the octahedral shape of the I@F-P NPs cannot be observed, which may be attributed to its destruction in lysosome through the clathrin-mediated endocytosis. Following this, the INS degradation rate of different preparations in the Caco-2 cell monolayers are reported. As shown in FIG. 13d, the ingested INS in the free INS group almost completely degrades after 48 h of incubation, while the I@P and I@F-P groups significantly inhibit the degradation rate of INS in PCFT high and low-expression cells. Compared with the I@P group, the FA slightly hinders the lysosome degradation and reduces the degradation rate from 60% to 48% in the PCFT low-expression cells. In contrast, only about 15% of INS degrades in PCFT high-expression cells, which is attributed not only to the protection of F-P but also the internalization of I@F-P through the Golgi-mediated pathway in, avoiding endocytosis by lysosomes and reducing INS degradation. In addition to these tests, the transcellular transport of I@F-P among different batches of cells is conducted using a previous protocol. As shown in FIG. 13e, the INS signal of I@F-P in high-expression cells is relatively high in batches (ii) and (iii), indicating that I@F-P achieves effective transcellular transport through the PCFT-mediated endocytosis. The above experiments proved that FA modification can increase the cellular uptake of I@F-P through the PCFT-mediated pathway in high-expressing cells, avoiding the degradation of lysosomal uptake, and thereby greatly promoting the transcellular transportation of INS.

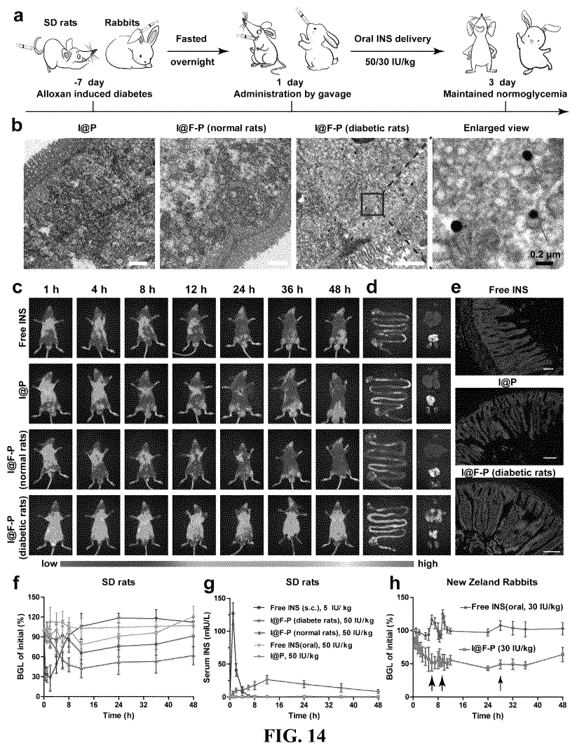

In vivo biodistribution and intestine absorption are investigated, particularly, the potential pathway and distribution of PCN-777-based nanosystems across the epithelial layer, the spatiotemporal distribution of the PCN-777-based nanosystem is mapped as it enters and exits the living body in detail using TEM, Maestro In Vivo Imaging System, and CLSM. Mice are sacrificed 4 hours after gavage, and the small intestine is taken for TEM imaging, as depicted in FIG. 14b. Only I@F-P NPs are localized in epithelial cells in large quantities, showing that PCFT-mediated endocytosis enhances the penetration efficiency of I@F-P NPs into intestinal epithelium. Subsequently, the in vivo images displaying the distribution of Free INS, I@P, and I@F-P in normal mice and diabetic mice are obtained within 48 hours. The results show a strong and persistent fluorescent signal up to 48 h is observed in the I@F-P-treated diabetic mice, while the fluorescent signal in other groups is weaker and continues to decrease within 48 h, as depicted in FIG. 14c, providing solid evidence for the efficient intestinal transportation and long-term efficacy of I@F-P in diabetic mice. It is worth noting that the observed fluorescence signal of I@F-P NPs in normal mice is nearly the same to that of the I@P group, indicating that the expression level of PCFT in normal mice is relatively low, thus leading to ineffective drug absorption.

Next, the biodistribution of INS in intestines and major organs after 12 h of administration is investigated, as depicted in FIG. 14 d. A strong fluorescent signal is detected only in the intestinal tract and liver of the I@F-P NPs group, demonstrating that only I@F-P NPs are efficiently absorbed by the intestinal tract and transported to the liver. Because the improved absorption of I@F-P NPs to diabetic mice is confirmed, the uptake of I@F-P NPs in intestine epithelial tissues after 12 h of administration is monitored to observe its distribution in the intestine more intuitively, as depicted in FIG. 14e. Fluorescent signals observed are localized within intestinal epithelial cells and muscular layer. Thus, confirming that INS from I@F-P has been successfully delivered into the intestinal villus, whereas only weak fluorescence between the intestinal villi gaps is observed in the free INS and I@P groups. The above results indicate that I@F-P NPs has an enhanced selective long-acting oral delivery function for diabetic mice.

In Vivo pharmacodynamics (PD) and pharmacokinetics (PK) are studied. The hypoglycemic response and pharmacokinetic effects of PCN-777-based nanosystems in fasted diabetic rats by gavage are undertaken. As shown in FIG. 14f, oral administration of free INS and I@P to diabetic rats and oral administration of I@F-P NPs to normal rats all fail to generate a significant glucose response, whereas subcutaneous injection of INS and intragastric administration of I@F-P NPs produce a rapid glucose response in diabetic rats. The difference between the above two hypoglycemic groups is that subcutaneous injection of insulin quickly restores high blood glucose levels (BGL) within 4 hours, while the I@F-P NPs group achieves the best hypoglycemic effect (BGL down to 45%) at 12 hours and maintains that level for 48 hours. The pharmacological bioavailability (PA %) of I@F-P NPs is about 38% based on the area above the curve (AAC) of the profile in FIG. 14f The corresponding serum INS profiles of rats treated with different formulations are depicted as the area under the curve (AUC_{0-48 h}) in FIG. 14g, and shows consistent results with regard to pharmacodynamics. The AUC of I@F-P is 782.3±126.3 mIU*h/mL with a relative bioavailability of 35.5% (Table S3). The ultrahigh bioavailability confirms that I@F-P enhanced oral insulin delivery with potent hypoglycemic function.

To further verify whether I@F-P can achieve similar effects in different animal species, oral I@F-P administration verification on New Zealand rabbits is conducted. I@F-P intragastrically is administered to fasting diabetic New Zealand rabbits and measured their BGL in real time. As depicted in FIG. 14h, the BGL is successfully reduced and maintained at normal levels for 48 hours even when rabbits are fed multiple times over different periods. In summary, I@F-P can exert an extraordinary and long-lasting hypoglycemic effect on multiple diabetic species (mice, rats, and rabbits), thus providing new evidence and implications for subsequent clinical research.

Conventional methods for the delivery of proteins/peptides with short half-lives suffer from cumbersome barriers negatively affecting their bioavailability and sustained release. These roadblocks have proven difficult to overcome and require that the proposed answer simultaneously address both needs in a single nanosystem. Based on previous work, which shows that acid-resistant Zr-based MOFs can achieve effective oral INS delivery, a new-generation oral INS delivery nanosystem based on FA-functionalized Zr-based MOFs (PCN-777) is constructed. Utilizing this, an excellent route for overcoming significant roadblocks by providing superior protection (kept 70% of INS active in trypsin buffer for 3 hrs), higher drug loading (up to 50 wt. %), extended-release dosing (of up to 48 hrs), higher bioavailability and the ability to traverse the complex physiological barriers present coupled with higher drug bioavailability. Thus, a more efficient and effective method than current injection techniques is identified. This is made possible by specifically coordinating functional molecules (FA) on the Zr₆ cluster of PCN-777. The final INS loaded nanosystem (I@F-P) NPs show selectively and effectively absorption by diabetic animals through the PCFT-mediated endocytic pathway. I@F-P NPs underwent efficient exocytosis through the Golgi pathway and maintain their octahedral shape in complete form during internalization for deeper penetration. Conversely, the I@F-P NPs are endocytosed in low efficiency through the clathrin-mediated pathway and become localized in the lysosome in normal animals with low expression of PCFT. The aforementioned advantages of the FA-conjugated PCN-777 nanosystem contribute to an oral insulin bioavailability as high as 35.5% in diabetic rats. Moreover, a similar hypoglycemic effect maintained for 48 hours is obtained in the diabetic New Zealand rabbit model. Meanwhile, no tissue lesions and abnormal biochemical indicators are observed in safety experiments after multiple administrations. This work indicates that the implementation of the FA-conjugated PCN-777 nanoplatform with its sustained-release capability can significantly improve patient compliance by reducing INS dosing frequency, eliminating the need for constant injections, providing consistency in drug release over the long term, maximizing drug utilization, and ameliorating medication safety. This simple and effective nanosystem integrating multiple benefits offers new possibilities for subsequent transformation toward clinical utilization.

Some embodiments have the following effects and advantages:
(1) the porous acid-resistant metal organic framework shows high drug-loading capacity for protein, strong protection, and good biocompatibility and slow and controlled release kinetics in vivo.
(2) The metal organic framework nano-particle for oral protein administration simultaneously overcomes the gastric juice barrier, the enzyme barrier and the intestinal epithelial cell barrier of oral protein, and can greatly improve the oral bioavailability of the protein.
(3) Some embodiments do not need to load enteric-coated materials, have low price, simple synthesis process and easy industrialization, and are expected to realize painless, controlled-release and convenient oral administration of protein.

A highly effective oral INS delivery system based on Tf-coated acid-resistant nMOFs (T@I@U) can be fabricated through a simple, low-cost, and easy scale-up process. The highly porous nMOFs demonstrates high protein drug loading capacity (up to about 33 wt. %) unattainable by most INS nanocarriers. By constructing T@I@U, INS is well protected by the nMOFs and Tf under the harsh environment in the GI tract and overcomes the gastric acid barrier, while exhibiting sustained and controlled release kinetics under physiological conditions. Meanwhile, modifying nMOFs allows Tf to be facilely installed on the well-defined surface to alter the nMOFs' penetration behaviors, providing synthetic advantages over those Tf-conjugated delivery nanosystems. The cellular experiments indicate that T@I@U first achieved effective apical endocytosis via the Tf-mediated uptake pathway, subsequently realizes rapid lysosome escape through the unique membrane switching effect, and releases from the cell monolayer eventually to overcome the basolateral membrane barrier. More significantly, the effectiveness of T@I@U by small animal research in which T@I@U quickly reduced BGL to around 30% of the initial level and prolonged the INS efficacy to 10 h is verified, bringing about a high oral bioavailability of 29.6%, which is about four times of that reported in Tf-decorated polymeric NPs. Besides, no tissue damage and abnormality of animals are observed in long-term experimentation, indicating the good biocompatibility of such an oral delivery nanosystem. Based on the above performance, the Tf-coated acid-resistant nMOFs are general oral protein delivery vehicles that can conquer various physiological obstacles in the GI tract. This nanosystem combines the advantages of efficient protein loading and protection, good intestinal permeability, controllable protein release, and a simple formulation strategy, for expanding the utility of protein-based oral therapies. In some embodiments, the effectiveness of this nanosystem can be further investigated in large animal species, in which the gastric emptying rate is slower and may aggravate the degradation of the nanosystem and the installed therapeutic or targeting protein. Furthermore, such a simple and effective MOF-based nanosystem can be expandable to integrate carefully selected therapeutic proteins with coated targeting proteins to create diverse functional nanosystems, providing the possibility to meet a wide range of biomedical applications.

Additional advantages may be apparent to one of skill in the art viewing this disclosure.

### EXAMPLES

The embodiments having been generally described, the following examples are given as particular embodiments of the disclosure and to demonstrate the practice and advantages thereof. It is understood that the examples are given by way of illustration and are not intended to limit the specification or the claims in any manner.

All standard synthesis reagents were purchased from commercial suppliers and used without any further purification. The chemicals ZrOCl₂·8H₂O, 2,5-dibromobenzenamine, 4-(methoxycarbonyl) phenylboronic acid, CsF, Pd(PPh₃)₄, TATB, FA, RITC, and FITC were obtained from Energy Chemical. The chemicals NaOH and CH₃COOH were purchased from Sinopharm chemical reagent. The materials INS, Tf, DAPI, lysosome tracker, Golgi tracker, and the BCA kit were obtained from Bio-sharp (Hefei, China). MTT and DMEM were purchased from Wuhan Kerui Biotechnology Co., Ltd. The bovine INS ELISA kit was obtained from Jiangsu Meimian industrial Co., Ltd.

To test the In vitro stability of UiO-68-NH₂-based nanosystems, different formulations were respectively dispersed in SGF, PBS, DMEM cell medium containing 10% FBS, and the size at various time points were measured by DLS.

To determine whether UiO-68-NH₂ could protect INS from digestive enzymes, the NPs were incubated in simulated intestinal fluid containing trypsin (1 mg/mL) at 37° C. Aliquots (100 μL) at specific time intervals were taken out and added 200 μL of DMSO containing 0.1% trifluoroacetic acid to stop the enzymatic reaction. The concentration of INS was determined with the Elisa kit in all the following experiments.

To test the release kinetic behaviours of I@U and T@I@U, samples were placed in dialysis bags (MWCO=10 KDa) and dispersed in ddH₂O, FaSSGF, FaSSIF (without phosphate), and PBS at 37° C. for 24 h. Additionally, the status of orally taken I@U and T@I@U was simulated by dialyzing the NPs against FaSSGF for 2 h, followed by FaSSIF for another 2 h and PBS (pH 7.4) for an additional 8 h. At different time points, 100 μL of buffer was taken, and an equal volume of fresh buffer was added to ensure a constant volume. The content of INS was determined using the BCA kit.

To study the bioactivity of INS after the preparation and drug release process, I@U and T@I@U were immersed in PBS for 24 h at 37° C. and isolated by ultra-centrifugation. The content of the released INS was determined using the BCA kit. Then the released INS (5 IU/kg) or free INS (5 IU/kg) was administered to the overnight fasting SD rats by subcutaneous injection.

The mouse blood samples were mixed with different concentrations of T@I@U to perform a hemolysis test in physiological saline solution (0, 0.125, 0.25, 0.5, 0.75, 1 mg/mL) and incubated at 37° C. for 4 h. The erythrocytes were mixed with H₂O in the positive group and saline in the negative group, respectively. After centrifugation, the UV absorption value of the supernatant at 540 nm was measured, and the hemolysis rate of the sample was calculated according to the formula:

Hemolysis=(Aₛₐₘₚₗₑₛ −A_{negative})/(Aₚₒₛᵢₜᵢᵥₑ −A_{negative})×100%.

For a cell culture, the human colon adenocarcinoma cell line (Caco-2) was purchased from the China Type Culture Collection. Caco-2 cells were cultured in a DMEM medium containing 20% fetal bovine serum (FBS) and 1% penicillin/streptomycin and placed in a 37° C., 5% CO₂ cell incubator.

Different cell lines at the density of 1×10⁴ cells/well were separately cultured in 96-well plates for studying cell cytotoxicity in vitro. A series of doses of UiO-68-NH₂, I@U, and T@I@U (0, 31.25, 62.5, 125, 250, 500 μg/mL) in the culture medium was applied to the cells for 24 h at 37° C. with 5% CO₂, and five samples were taken in parallel. The medium was discarded, and the cells were exposed to freshly prepared MTT solution (5 mg/mL) for another 4 h. Afterward, DMSO (100 μL) was added to wells. The optical density (OD) was monitored at 490 nm using Microplate Reader.

Caco-2 cells were seeded in 12-well plates and cultured for 3 days for a live and dead cell viability assay. The cells were treated with DMEM medium containing different samples for 24 h, and the live/dead cells were stained with calcium AM and PI. The images were taken by an inverted fluorescence microscope.

To visualize the uptake of UiO-68-NH₂, I@U, and T@I@U for cellular internalization studies in vitro, Caco-2 cells were cultured in twelve-well plates with slides for three days and incubated with either free RITC-INS, RITC-INS@U or T@RITC-INS@U at 37° C. for different time intervals. Subsequently, the cells were fixed with 4% paraformaldehyde and stained with DAPI for 20 min. After rinsing the slides with PBS, the location of NPs in cells was observed by CLSM (LSM 800, Zeiss, Germany). The intensity of fluorescence was also measured by flow cytometry.

To further evaluate the role of Tf in the uptake trial, Caco-2 cells were incubated with double-labeled T@I@U. In addition, the cellular uptake of T@I@U was studied with prior incubation with Tf. After incubation with different formulations for 2 h at 37° C., the amounts of RITC-INS were determined using CLSM.

The co-localization of nMOFs and lysosomes was characterized. In brief, T@RITC-INS@U were added to the cells and incubated at 37° C. for various time intervals (15, 30, 60, 120, 240 min). Subsequently, Caco-2 cells were stained with Lyso-Tracker Green for 30 min. The CLSM was used to analyze the co-localization of the samples and lysosomes.

The deep penetration of T@I@U was studied as the literature reported. Caco-2 cells on coverslips (i) were cultured in a medium that contained different INS formulations for 4 h. Then coverslip (i) was put into fresh culture medium and fresh cells on the coverslip (ii) for 12 h. The above step was repeated to obtain the coverslip (iii). The coverslips (i, ii, iii) were visualized by CLSM separately.

The Caco-2 cells were seeded into a transwell insert and cultured for three weeks till the cell monolayer membrane was formed (the TEER values reached above 500Ω×cm²) for an in vitro transepithelial transport. Free INS and I@U and T@I@U in the medium were added to the apical side. Samples were taken out from the basolateral side at 0, 0.083, 0.25, 0.5, 1, 1.5, 2 h, and the medium was always supplemented to keep the volume consistent. During the process, the TEER values were constantly measured to determine the integrity of tight junctions. The INS contents were measured using the bovine insulin ELISA kit. The Papp values of INS from the different treatments were calculated as reported.

Animal models using male Sprague Dawley rats (180-200 g) and BALB/c mice (18-20 g) were provided by Beijing Vital River Laboratory Animal Technology Co. Ltd. All the animal studies were conducted following the Chinese Regulations for the Administration of Affairs Concerning Experimental Animals and performed in compliance with the guidelines of the Institutional Animal Care and Use Committee (IACUC) of Wuhan University.

The intestinal penetration experiment in vitro was carried out according to an isolated intestine penetration test. The SD rats were fasted overnight and then sacrificed. About 5 cm of duodenum, jejunum, and ileum with both ends ligated were taken out, and then 200 μL of free INS, I@U, and T@I@U (10 IU/ml) were injected into the intestinal lumen. The intestine was then incubated in 5 mL PBS buffer at 37° C. with gentle stirring. The samples were collected according to the above method, and the amount of INS released was again quantified by ELISA kit.

RITC-INS, RITC-INS@U, T@RITC-INS@U were orally administered to the overnight fasted mice for in vivo fluorescence imaging. The fluorescence images of mice were captured at different time points using the SI Imaging Amix imaging system to evaluate the metabolic process of INS preparations. Another group of mice was sacrificed after 4 h of treatment. The images of organs and intestines were also obtained. Different formulations were orally administered to the overnight fasted rats for 4 h. Then the rats were sacrificed, and the intestine loops were excised and frozen in Tissue-Tek OCT with liquid nitrogen. Cross intestine sections were obtained using a freezing microtome blade and then stained the intestine sections with DAPI for 10 min and visualized by CLSM.

In vivo INS bioactivity studies were performed in alloxan-induced diabetic animals (n=six per group). The rats and mice were induced to type I diabetes by alloxan injections. The animals were regarded as diabetic if their fasting blood glucose levels were higher than 11 mmol/L in one week after injection.

The rats and mice fasted overnight before experiments and then administered different samples by gavage. The hyperglycemic effect was tested following the oral administration of I@U and T@I@U (20 IU/kg). As control groups, one group of rats were administered free INS (20 IU/kg) by oral gavage, and the other group was administered free INS (5 IU/kg) by s.c. injection. Blood samples were collected from the orbital venous plexus and analyzed using a blood glucose meter, expressed as the percentage of baseline serum glucose level (0 h) at different periods. The INS contents in blood samples were measured using the bovine INS ELISA kit. The 0 h INS content was subtracted from the INS measured at different time intervals and plotted.

Mice received oral gavage with free INS, I@U, and T@I@U twice a day for seven consecutive days for histologic examination. The mice were sacrificed, and their organs and intestines were put in 4% (w/v) formalin. Then the organs were embedded in paraffin, and the tissue was cut into slices, both of which were later stained with hematoxylin and eosin (H&E) for microscopic examination.

Statistical significance was determined by statistical analysis using two-tailed Student's t-test. P less than 0.05(*) was considered statistically significant. Unless otherwise noted, all parallel experiments are conducted in three groups (n=three). Error bars used in this work were SD.

In some embodiments, the preparation and characterization of PCN-777, I@P, and I@F-P was undertaken. PCN-777 was synthesized through the solvothermal method according to modifying methods. In brief, ZrOCl₂·8H₂O (52 mg), TATB (13 mg), and trifluoroacetic acid (0.24 mL) in DMF (5 mL) were ultrasonically dissolved in a 20 mL Pyrex vial. The mixture was kept stirred at 120° C. for 0.5 h. After cooling to room temperature, PCN-777 was collected by centrifuging the mixture and washing it three times with DMF and methanol, respectively. An amount of 200 μL solution was dried to a powder and weighed for quantification. For the preparation of F-P, 1 mL folate acid (FA) (2 mg/mL in DMF) was added to 5 mL PCN-777 (2 mg/mL in DMF) and vigorously stirred for 12 h. Different weight ratios between FA and PCN-777 (FA/PCN weight ratio of 1:10, 1:5, and 2:5) were separately investigated to optimize the loading parameters. Next, the mixture was centrifuged (11,000 rpm, 10 min.) and washed three times with DMF to remove excessive free FA ligands. The amount of FA installed on the PCN-777 surface is quantified by reading the characteristic absorption band of FA at 280 nm using UV-Vis. Subsequently, 1 mL of INS solution (10 mg/mL, dissolved in dilute HCl, pH=2.0) was added dropwise to 20 mL of FA-PCN-777 dispersion (0.5 mg/mL in ddH₂O) and stirred for 12 h at room temperature to obtain I@F-P. Different feeding ratios (INS: PCN-777=3:1, 2:1, 1:1, 1:2, 1:3) were also investigated. The resulting mixture was purified by centrifugation. The encapsulation efficiency of INS was found to exceed 50% by quantifying the insulin content retained in FA-modified PCN-777 using BCA protein assay and thermogravimetric analysis (TGA, TGA 50).

The obtained PCN-777, F-P, I@P, and I@F-P were characterized by Malvern Zetasizer Nano series ZS-90 to measure the hydrodynamic size and zeta potential. The TEM tests were performed using a HITACHI H-7000FA TEM to evaluate the morphology of the samples. The EDS elemental mapping analysis was performed by FEI Talos F200X. PXRD was carried out with a Rigaku MiniFlex 600 X-ray diffractometer under Cu Kα radiation (parameters: 600 W). The UV-Vis spectroscopy was measured by a UV-Vis spectrophotometer (UV-2600, Shimadzu, Japan). The fluorescence signal of the PCN-777-based nanosystems was detected by a fluorescence spectrophotometer (RF-6000, Shimadzu, Japan). Laser confocal fluorescence microscope (CLSM) images were collected by Carl Zeiss NOL-LSM 710.

FA loading capacity was calculated by the following equation:

LC=(total weight of FA−weight of supernatant FA)/weight of PCN-777×100%

INS loading capacity was calculated by the following equation:

LC=(total weight of INS−weight of supernatant INS)/weight of PCN-777×100% INS

loading content was calculated by the following equation:

LC=(total weight of INS−weight of supernatant INS)/(weight of PCN-777+total weight of INS−weight of supernatant INS+total weight of FA−weight of supernatant FA)×100%

Procedures were undertaken for In vitro stability. The procedures to quantify the PCN-777-based nanosystem stability and INS degradation by the enzyme were conducted as described previously. The different formulations were respectively dispersed in ddH₂O, DMEM cell medium containing 10% FBS, and the size at various time points was measured by DLS. As for the protective performance determination of PCN-777-based nanosystem on insulin in enzyme, the NPs were incubated in simulated intestinal fluid containing trypsin (1 mg/mL) at 37° C. for 3 h. At different time intervals, aliquots of 100 μL were taken out and 200 μL of DMSO containing 0.1% trifluoroacetic acid was added to quench the enzymatic reaction. The NPs were then completely disrupted using PBS and the concentration of INS was determined via a bovine INS Elisa kit.

To investigate whether the bioactivity of INS was maintained after preparation, long-term storage, and drug release, I@P, and I@F-P after two months of synthesis were immersed in PBS at 37° C. for 48 hours and then separated by ultracentrifugation. The amount of INS released in the supernatant was determined using a BCA protein assay kit. Released INS (5 IU/kg) or free INS (5 IU/kg) was then administered to overnight fasted SD rats by subcutaneous injection.

A study of release kinetics is undertaken. To test the release profiles of INS from I@P and I@F-P, different INS-loaded formulations were placed in dialysis bags (MWCO=10 KDa) and dispersed in FaSSGF, FaSSIF (without phosphate), and PBS at 37° C. for 48 h. At different time points, 100 μL of buffer solution was taken for testing, and an equal volume of fresh buffer solution was added to keep the volume constant. The quantity of INS was determined using the BCA protein assay kit. Furthermore, the physiological environment of I@P and I@F-P was simulated after oral administration by dialyzing the NPs in FaSSGF for 2 h, and then in FaSSIF for another 2 h and PBS (pH 7.4) for an additional 44 h. Sampling and INS concentration detection were carried out as described in the disclosed experimental procedures. In addition, TEM images of I@P and I@F-P incubated was taken with PBS for different time intervals to visualize the disintegration of the NPs.

In some embodiments, a hemolysis test was undertaken with rats. Rat blood samples were incubated with different concentrations of I@F-P in saline solution (0.05, 0.1, 0.2, 0.3, 0.5 mg/mL) at 37° C. for 4 h. The groups in which erythrocytes were incubated with H₂O and saline were set as the positive control (PC) and negative control (NC). After 4 h of incubation, the samples were centrifuged and measured the supernatant was at 540 nm using the UV-vis, and the hemolysis rate of the samples was calculated using the following equation:

$$Hemoly sis = ( {A}_{samples} - {A}_{negative} ) / ( {A}_{positive} - {A}_{negative} ) \times 100 \% .$$

For a cell culture, the human colon adenocarcinoma cell line (Caco-2), murine colorectal carcinoma cell line (CT-26), and human normal liver cell line (L02) were purchased from the China Type Culture Collection. CT-26 cells and L02 cells were cultured in Dulbecco's Modified Eagle's medium (DMEM) supplemented with 10% fetal bovine serum (FBS)(20% required for Caco-2) and 1% penicillin/streptomycin in an atmosphere of 5% CO₂ at 37° C.

In vitro cytotoxicity and live and dead assays were undertaken. In vitro cytotoxicity was assessed using an MTT assay in Caco-2 cells, CT-26 cells, and L02 cells, as well as a live and dead cell assays. Different cell lines were separately cultured in 96-well plates at the density of 1×10⁴ cells and well. The cells were then incubated with 100 μL of PCN-777, I@P, and I@F-P at concentrations of 0, 31.25, 62.5, 125, 250, and 500 μg/mL in DMEM, respectively, and five samples were taken in parallel. After 24 h of incubation, the cells were exposed to 100 μL freshly prepared MTT solution (5 mg/mL) for another 4 h. Afterward, the supernatant was carefully removed and DMSO (100 μL) was added to each well. The optical density (OD) was measured at 490 nm using Microplate Reader. The live and dead assays were performed by staining Caco-2 cells following PCN-777-based nanosystems (500 μg/mL) administration with 100 μl of PBS buffer containing calcium AM and PI. Finally, the cells were observed by an inverted fluorescence microscope.

Cellular internalization in vitro studies are undertaken. Caco-2 cells were seeded in twelve-well plates with slides at a density of 1×10⁵ cells per well overnight and subsequently treated with or without myricetin solution (150 mmol/L) similar to the PCFT low expression group or high expression group in the earlier reported procedure. To estimate the internalization of NPs in the PCFT high- and low-expression groups, Caco-2 cells or myricetin pre-treated Caco-2 cells were incubated with free RITC-INS, R-I@P, and R-I@F-P with different modification densities of FA (hereinafter referred to as I@F-P (1:10, 1:5, 2:5)) at 37° C. for different time intervals. The cells were further fixed with 4% paraformaldehyde and stained with DAPI for 20 min. The cell internalization of PCN-777-based nanosystems was visualized by CLSM (LSM 800, Zeiss, Germany) and was also quantitively measured using flow cytometry. The three-dimensional visualization was conducted by the Z-stack method. To further identify the uptake mechanisms of I@F-P in the PCFT high- and low-expression groups, Caco-2 cells were treated with RITC-labelled I@F-P under hypothermia or co-incubated with various inhibitors (lovastatin (1 mg/mL), methyl-b-cyclodextrin (10 mmol/L), filipin (1 mg/mL), chlorpromazine (10 mg/mL), cytochalasin D (1 mg/mL)) at 37° C. After incubation for 4 h, the internalized amounts of RITC-INS were visualized by CLSM and quantified using flow cytometry.

To elucidate the intracellular trafficking of NPs, Caco-2 cells were incubated with I@P and I@F-P (1:10, 1:5, 2:5) for 4 h at 37° C. Subsequently, Caco-2 cells were stained with Lyso-Tracker Green or Golgi-tracker Green for 30 min. Colocalization of samples and lysosomes/Golgi was detected using CLSM.

Transepithelial transport studies are being undertaken. The transepithelial transport studies were conducted following previously reported procedures. In short, the Caco-2 cells were cultured in transwell inserts for 21 days till the TEER values reached above 300Ω×cm². Caco-2 cell monolayers with high or low expression of PCFT were co-incubated with Free INS and I@P and I@F-P on the apical side for 48 hours. An amount of 100 μL of the sample was taken out from the basolateral side at different time intervals and 100 μL of DMEM was always supplemented to maintain a consistent volume. After 48 hours of incubation, samples of the apical and basolateral layers were collected respectively, and the cell monolayer was lysed with RIPA lysate and collected. The INS contents in each sample were then tested utilizing the bovine insulin ELISA kit. The degradation rate of INS in different preparations were calculated according to the following equation:

$$Degradation \% = \frac{m ( total ) - m ( apical ) - m ( basolateral ) - m ( inside )}{m ( total ) - m ( basolateral )} \times 100 \%$$

where m(total) is the total mass of INS added, m(apical) is the mass of INS in the apical layer, m(basolateral) is the mass of INS in the basolateral layer, and m(inside) is the mass of INS in the cell monolayer.

To intuitively illustrate the efficiency of transcellular transport of PCN-777-based nanosystems, RITC-labeled INS preparations are used to co-incubate cell monolayers and SI Imaging Amix imaging system to observe the fluorescence distribution on the apical and basolateral side. TEER values were measured at various time points to determine tight junction integrity during the drug administration process. The following equation was utilized to calculate the apparent permeability coefficient (Papp) from the different treatments as in the following equation:

$${P}_{app} = \frac{dQ}{dt} \times \frac{1}{A \times {C}_{0}}$$

where dQ/dt is the flux of INS from the apical side to the basolateral side, A is the membrane area (cm²), and C₀ is the initial concentration of NPs on the apical side.

In addition, the deep penetration of I@F-P between different cell batches was previously studied. The three different cell batches were observed by CLSM. Next, to verify that the NPs maintained its intact morphology after penetrating the cell monolayer via a Golgi-mediated pathway, samples of I@P and I@F-P were collected from the basolateral side at 24 h and obtained TEM images after centrifugation.

Animal models and diabetes induction were undertaken. Male Sprague Dawley rats (180-200 g) and BALB/c mice (18-20 g) were provided by Beijing Vital River Laboratory Animal Technology Co. Ltd. New Zealand rabbits (2-2.5 kg) were provided in all the animal studies were conducted following the Chinese Regulations for the Administration of Affairs Concerning Experimental Animals and performed in compliance with the guidelines of the Institutional Animal Care and Use Committee (IACUC) of Wuhan University. To establish an alloxan-induced diabetic rat model, healthy rats were intravenously injected with alloxan 50 mg/kg once and the blood glucose changes were monitored daily to confirm their diabetic status. After seven days, the blood glucose was stable at 20 mmol/L, proving the diabetes model was successfully established. The difference between the establishment of diabetes models of mice and rabbits was that the dosage of alloxan in mice and rabbits is 100 mg/kg. If the blood glucose level of rabbits did not rise after the first injection, 100 mg/kg of alloxan would be injected again through the ear vein 48 hours later.

In vivo fluorescence imaging is undertaken. RITC-INS, RITC-INS@P, and R-I@F-P were orally administered to the overnight fasted diabetic mice or normal mice. The SI Imaging Amix imaging system was used to assess the distribution and metabolism of INS preparations in mice with different intestinal expressions of PCFT at different time points. Another group of diabetic mice or normal mice was sacrificed twelve hours after RITC-INS, RITC-INS@P, and R-I@F-P treatment, and the organs and intestines were collected and observed. The same procedure was done to diabetic rats. The intestine loops of the RITC-INS, RITC-INS@P, and R-I@F-P medicated rats were collected by the same procedure and frozen in Tissue-Tek OCT with liquid nitrogen. Intestinal sections were obtained using a cryostat and visualized by CLSM after staining with DAPI for 10 min.

Tracking nanoparticle diffusion in the small intestine is analyzed. The rats were sacrificed four hours after oral administration of different preparations, and the small intestines were collected and washed with PBS. Samples were serially fixed in a solution of 2.5% glutaraldehyde and 2.5% paraformaldehyde in 0.1M PBS buffer and 4% osmium tetroxide solution and then transferred to a Beem embedding capsule. Micro-embedding resin was added to the embedded samples to obtain blocks, which were further sectioned with ultramicrotomy, and these sections were retrieved through a copper grid and then visualized under TEM.

INS bioactivity in vivo was undertaken. In vivo studies were performed in alloxan-induced diabetic rats (n=five per group) and rabbits (n=three per group). The rats and rabbits were fasted overnight before experiments and then administered free INS by gavage (50 IU/kg) and by subcutaneous injection (5 IU/kg). At predetermined time intervals, blood samples were collected for glucose testing using a blood glucose meter. Serum insulin level was determined using a bovine INS ELISA kit. During the experiment, the rats drank water arbitrarily and were fed at 12 hours and 36 hours, and the rabbits were fed at 5 hours, 10 hours, 12 hours, 28 hours, and 36 hours, respectively.

Long-term safety of consecutive dosing was determined. Mice received oral gavage with Free-INS, I@P, and I@F-P once every two days for fourteen consecutive days. Mice were then sacrificed after blood collection (for routine blood and blood biochemical indicators measurement), and organs and intestines were soaked in 4% (w/v) formalin and then embedded in paraffin. The tissue was sectioned and stained with hematoxylin and eosin (H&E) for microscopic examination.

Statistical analysis was determined by a two-tailed Student's t-test using the software Prism. P less than 0.01(**), P less than 0.001(***) was considered statistically significant. All of the results presented were means±S.D.

### Example 1

Preparation and characterization of oral insulin metal organic framework nanoparticles (T @ I @ U)

Amino-triphenyl dicarboxylic acid (amino-TPDC) was synthesized according to the modifying methods as previously reported. Preparation of UiO-68-NH by solvothermal method 2: amino-TPDC (10.5 mg, 0.031 mmol) and ZrOCl₂·8-H₂O (8.3 mg, 0.026 mmol) was dissolved in 2 mL DMF. The mixture was sonicated for 5 minutes until it was clear and transparent. To the mixture was added 40 μL of acetic acid, followed by heating at 90° C. for 6 hours. The mixture was centrifuged at 11,000 rpm and washed three times with DMF to obtain purified UiO-68-NH₂. Centrifugation was continued at 11,000 rpm for three exchanges of DMF to ddH₂O. A 100 μL solution was dried to powder and weighed for quantification.

For the preparation of I@U, an amount of 50-300 μL, such as 200 μL, of insulin solution (20 mg/mL, pH 2.0, dilute hydrochloric acid as solvent) was added dropwise to 4 mL of freshly synthesized UiO-68-NH₂ dispersion (0.25 mg/mL in ddH₂O) with stirring at room temperature (0.25 mg/mL) in water, stirring was continued for 12 hours to obtain insulin @ metal organic framework nanoparticles (I @ U). Subsequently, 40 μL of an aqueous transferrin solution (5 mg/mL) was added to the above solution and stirring was continued for 12 hours to obtain transferrin @ insulin @ metal organic framework nanoparticles (T @ I @ U), and the unencapsulated protein was removed by low temperature centrifugation at 11,000 rpm at 4° C. The drug loading of the protein was determined using the BCA method. As depicted in FIG. 1, the metal organic framework greatly improves the drug loading of insulin when the islets of Langerhans when the feeding mass ratio of the insulin to the metal organic framework reaches (4:1), the drug loading of the insulin reaches 33 percent. The INS loading content was measured by the BCA kit and thermogravimetric analysis (TGA). INS loading content was calculated by:

$$Loading content = ( total weight of drug - weight of drug in supernatant ) / weight of drug - loaded NPs \times 100 \%$$

Thus, I @ U, T @ I @ U, insulin and metal organic frameworks used in the examples which follow, were all 4:1 in mass ratio.

Zeta potential and hydrodynamic size of UiO-68-NH₂, I@U, and T@I@U were measured by Malvern Zetasizer Nano series ZS-90. The TEM tests were performed by HITACHI H-7000FA TEM to evaluate the morphology of the samples. The EDS elemental mapping analysis was performed by FEI Talos F200X. PXRD was carried out with a Rigaku MiniFlex 600 X-ray diffractometer under Cu Kα radiation (parameters: 600 W). The UV-Vis spectroscopy was measured by a UV-Vis spectrophotometer (UV-2600, Shimadzu, Japan). The fluorescence signal of the nanosystems was detected by a fluorescence spectrophotometer (RF-6000, Shimadzu, Japan). The thermogravimetric analysis (TGA) was observed by TGA 50. Laser confocal fluorescence microscope (CLSM) images were collected by Carl Zeiss NOL-LSM 710.

Subsequently, the particle size and morphology of I @ U and T @ I @ U were examined by transmission electron microscopy, as shown in FIG. 2, with little change in the morphology and particle size of the NPs after loading with insulin. After the transferrin is physically adhered to the surface of I @ U, the shape of the nanoparticle is gradually changed from a regular octahedron to a sphere, and the particle size is increased. The above results indicate that oral insulin metal organic framework nanoparticles were successfully synthesized.

### Example 2

### Insulin Activity Assay.

To verify that insulin still has comparable biological activity during the preparation and release of the nanoformulations, in this example, I @ U and T @ I @ U, freshly prepared as in example 1, were immersed in 1 mL of PBS buffer at 37° C. for 24 hours and centrifuged at low temperature and ultra-speed (11,000 rpm) to obtain a supernatant. The content of released insulin in the supernatant was determined using the BCA kit, and then the released insulin (5 IU/kg) and the insulin without drug loading (5 IU/kg) were administered to the overnight fasted SD rats, respectively, by subcutaneous injection. The blood glucose concentration of rats was measured at different time points. The results are shown in FIG. 3, which shows that the prepared insulin metal-organic framework nano-particles do not damage the biological activity of insulin in the preparation process.

### Example 3

### Study of the Protective Properties of T @ I @ U on Insulin

In order to determine UiO-68-NH₂ and whether insulin can be protected from digestive enzymes I @ U and T @ I @ U, samples were freshly prepared as in Example 1 and were incubated in HBSS buffer with trypsin (1 mg/mL) at 37° C. Aliquots (100 μL) were removed at specified time intervals, 200 μL of DMSO containing 0.1% trifluoroacetic acid was added to stop the enzymatic reaction, and the concentration of insulin in the solution was determined using an Elisa kit. As shown in FIG. 4, I @ U and T @ I @ U protect insulin well within 2 hours.

### Example 4

### Release Kinetics of T @ I @ U in Different Release Media

I @ U and T @ I @ U were placed in dialysis bags (MWCO=10 KDa) and dispersed in double distilled water (ddH₂O) at 37° C. respectively, FaSSGF, FaSSIF (without phosphate) and PBS for 24 hours, 100 μL of buffer was taken at different time points, and an equal volume of fresh buffer was added to ensure constant volume. The content of insulin in the taken buffer was measured using the BCA kit, and as a result, as shown in FIG. 5, T @ I @ U was hardly released from the simulated gastrointestinal tract solution, whereas insulin released from UiO-68-NH₂ under PBS conditions was released slowly and almost completely within 12 hours. Experimental results show that the prepared T @ I @ U has the characteristic of controlled release.

### Example 5

### Ex Vivo Intestinal Permeation Test

SD rats were fasted overnight and then sacrificed. The duodenum, jejunum and ileum were removed 5 cm each and the ends were ligated. Next, 200 μL of insulin, I @ U and T @ I @ U (10IU/mL) were injected into the intestinal lumen. The intestine was then incubated in 5 mL PBS buffer at 37° C. with gentle stirring. At different time points, 100 μL of buffer was taken and an equal volume of fresh buffer was added to ensure constant volume. The amount of insulin released was quantified using an ELISA kit. The results are shown in FIG. 6, where T @ I @ U significantly increased the permeability of insulin in the intestinal tract via transferrin-mediated transport pathway, enabling oral delivery of insulin.

### Example 6

### Example 6 In Vivo Pharmacodynamic and Pharmacokinetic Studies of T @ I @ U

Hypoglycemic effects (FIG. 7A, B) and insulin levels in the serum of SD rats (FIG. 7C) were tested after administration of equal amounts of insulin I @ U and T @ I @ U (20IU/kg) to diabetic mice (BALB/C mice) and rats (SD rats) by gavage. As a control group, one group of rats was administered insulin (20 IU/kg) by gavage, and the other group was administered insulin (5 IU/kg) by subcutaneous injection. Blood samples were collected from the orbital venous plexus and the hypoglycemic effects of the different samples were determined using a glucometer, the results of which are shown in FIG. 7. It was shown that T @ I @ U rapidly reduced blood glucose levels to around 30% of the initial level and extended insulin potency to 10 hours, resulting in high oral bioavailability.

Having described various systems and methods herein, certain embodiments can include, but are not limited to:

In a first aspect, a composition comprises: a metal-organic framework (MOF) nanoparticle for oral protein administration, wherein the MOF comprises a nano-scale acid-resistant MOF; a small molecular protein/polypeptide drug and a targeting molecule, wherein the MOF is loaded with the small molecular protein/polypeptide; and targeting molecules, wherein a surface of the MOF is coated with the targeting molecules.

A second aspect can include the composition of the first aspect, wherein the MOF is UiO-68-NH₂, PCN-222, PCN-224, or any combination thereof.

A third aspect can include the composition of the first aspect or second aspect, wherein the small molecular protein/polypeptide is a protein/polypeptide with a molecular weight less than or equal to about 10,000, about 9,000, about 8,000, about 7,000, about 6,000, or about 5,000 Dalton and has therapeutic effect comprising salmon calcitonin, glucagon, interferon, or growth hormone.

A fourth aspect can include the composition of any one of the first to third aspects, wherein the targeting molecules can target receptors on intestinal epithelial cell membranes, comprising transferrin, neonatal Fc receptor protein, cell penetrating peptide, octaarginine, or any combination thereof.

A fifth aspect can include the composition of any one of the first to fourth aspects, wherein the MOF is UiO-68-NH₂, the small molecular protein/polypeptide is insulin, and the targeting molecules are transferrin.

A sixth aspect can include the composition of the fifth aspect, wherein the MOF is UiO-68-NH₂, the mass ratio of the insulin to the MOF is about 1:1-about 1:6, about 1:1-about 1:5, or about 1:1-about 1:3, UiO-68-NH₂, and the mass ratio of the transferrin to the MOF is about 1:0.1-about 1:1, about 1:0.3-about 1:1, or about 1:0.5-about 1:1.

A seventh aspect can include a method of preparing metal organic framework (MOF) nanoparticles for oral protein administration according to any one of the first to sixth aspects, the method comprises: adding the micromolecule protein/polypeptide or the solution thereof into the acid-resistant metal organic framework water solution; continuously stirring to ensure that the acid-resistant metal organic framework is coated with the micromolecule protein/polypeptide; and adding the targeting molecules or the solution thereof, and continuously stirring to ensure that the targeting molecule is adhered to the acid-resistant metal organic framework to obtain the metal organic framework nano-particle for oral protein administration.

An eighth aspect can include the method of the seventh aspect, wherein the preparation method is carried out at about 4-about 35° C., about 10-about 25° C., or about 15-about 20° C.

A ninth aspect can include the method of the seventh aspect or the eighth aspect, wherein the acid-resistant metal organic framework is UiO-68-NH₂ when the small molecular protein/polypeptide is insulin and the targeting molecule is transferrin, the preparation method comprises the following steps dissolving insulin powder in dilute hydrochloric acid solution with a pH of about 1.0-about 3.0 or about 2.0, and dropwise adding the insulin hydrochloric acid solution to UiO-68-NH₂ under stirring at room temperature; adding the mixture into the aqueous solution and continuously stirring for about 0.1 to about 12 hours, about 1.0 to about 10 hours, or about 3 to about 8 hours; and under the condition of stirring at room temperature, adding the transferrin solution and continuously stirring for about 0.1-about 12 hours, about 1.0 to about 10 hours, or about 3 to about 8 hours, wherein the unencapsulated or unattached proteins are removed by centrifugation.

In a tenth aspect, a composition comprises a metal-organic framework (MOF) nanoparticle for oral protein administration, wherein the MOF comprises a nano-scale acid-resistant MOF; a small molecular protein/polypeptide drug and a targeting molecule, wherein the MOF is loaded with the small molecular protein/polypeptide; and targeting molecules, wherein a surface of the MOF is coated with the targeting molecules.

An eleventh aspect can include the composition of the tenth aspect, wherein the MOF nanoparticle comprises a conjugated mesoporous acid-resistant MOF.

A twelfth aspect can include the composition of the tenth or eleventh aspect, wherein the MOF comprises a Zr-MOF.

A thirteenth aspect can include the composition of any one of the tenth to twelfth aspects, wherein the MOF is PCN-777.

A fourteenth aspect can include the composition of any one of the tenth to thirteenth aspects, wherein the small molecular protein/polypeptide is a protein/polypeptide with a molecular weight less than or equal to about 10,000, about 9,000, about 8,000, about 7,000, about 6,000, or about 5,000 Dalton and has therapeutic effect comprising salmon calcitonin, glucagon, interferon, or growth hormone.

A fifteenth aspect can include the composition of any one of the tenth to fourteenth aspects, wherein the targeting molecules can target receptors on intestinal epithelial cell membranes, comprising transferrin, neonatal Fc receptor protein, cell penetrating peptide, octaarginine, or any combination thereof.

A sixteenth aspect can include the composition of any one of the tenth to fifteenth aspects, wherein the MOF is PCN-777, the small molecular protein/polypeptide is insulin, and the targeting molecules comprise a folate.

A seventeenth aspect can include the composition of any one of the tenth to sixteenth aspects, wherein the targeting molecules comprise folic acid.

An eighteenth aspect can include the composition of any one of the tenth to seventeenth aspects, wherein: the MOF is PCN-777, the targeting molecule is folic acid, and the mass ratio of the folic acid to the PCN-777 is between about 1:10 to about 4:10, or about 2:10 to about 3:10.

In a nineteenth aspect, a method of preparing metal organic framework (MOF) nanoparticles for oral protein administration, the method comprises adding the micromolecule protein/polypeptide or the solution thereof into an acid-resistant metal organic framework solution; continuously stirring to ensure that the acid-resistant metal organic framework is coated with the micromolecule protein/polypeptide; and adding the targeting molecules or the solution thereof, and continuously stirring to ensure that the targeting molecule is adhered to the acid-resistant metal organic framework to obtain the metal organic framework nano-particle for oral protein administration.

A twentieth aspect can include the method of the nineteenth aspect, wherein the preparation method is carried out at about 80-about 130° C., about 90 to about 120° C., or about 100 to about 110° C.

A twenty-first aspect can include the method of the nineteenth aspect or twentieth aspect, wherein the acid-resistant metal organic framework is PCN-777 when the small molecular protein/polypeptide is insulin and the targeting molecule is folic acid.

In a twenty-second aspect, a method of treating a subject with diabetes comprises: administering a therapeutically effective amount of a composition to a subject in need thereof, wherein the therapeutically effective amount is administered as an oral composition, and wherein the oral composition comprises: a metal-organic framework (MOF) nanoparticle for oral protein administration, wherein the MOF comprises a nano-scale acid-resistant MOF; a small molecular protein/polypeptide drug and a targeting molecule, wherein the MOF is loaded with the small molecular protein/polypeptide; and targeting molecules, wherein a surface of the MOF is coated with the targeting molecules.

A twenty-third aspect can include the method of the twenty-second aspect, wherein at least about 35%, about 45%, or about 55% of the small molecular protein/polypeptide is released within 48 hours of administration.

A twenty-fourth aspect can include the method of the twenty-second aspect or the twenty-third aspect, wherein the MOF nanoparticle comprises a conjugated mesoporous acid-resistant MOF.

A twenty-fifth aspect can include the method of any one of the twenty-second to twenty-fourth aspects, wherein the MOF comprises a Zr-MOF.

A twenty-sixth aspect can include the method of any one of the twenty-second to twenty-fifth aspects, wherein the MOF is PCN-777.

A twenty-seventh aspect can include the method of any one of the twenty-second to twenty-sixth aspects, wherein the small molecular protein/polypeptide is a protein/polypeptide with a molecular weight less than or equal to about 10,000, about 9,000, about 8,000, about 7,000, about 6,000, or about 5,000 Dalton and has therapeutic effect comprising salmon calcitonin, glucagon, interferon, or growth hormone.

A twenty-eighth aspect can include the method of any one of the twenty-second to twenty-seventh aspects, wherein the targeting molecules can target receptors on intestinal epithelial cell membranes, comprising transferrin, neonatal Fc receptor protein, cell penetrating peptide, octaarginine, or any combination thereof.

A twenty-ninth aspect can include the method of any one of the twenty-second to twenty-eighth aspects, wherein the MOF is PCN-777, the small molecular protein/polypeptide is insulin, and the targeting molecules comprise a folate.

A thirtieth aspect can include the method of any one of the twenty-second to twenty-ninth aspects, wherein the targeting molecules comprise folic acid.

A thirty-first aspect can include the method of any one of the twenty-second to thirtieth aspects, wherein: the MOF is PCN-777, the targeting molecule is folic acid, and the mass ratio of the folic acid to the PCN-777 is between about 1:10 to about 4:10, or about 2:10 to about 3:10.

For purposes of the disclosure herein, the term “comprising” includes “consisting” or “consisting essentially of.” Further, for purposes of the disclosure herein, the term “including” includes “comprising,” “consisting,” or “consisting essentially of.”

Accordingly, the scope of protection is not limited by the description set out above but is only limited by the claims which follow, that scope including all equivalents of the subject matter of the claims. Each and every claim is incorporated into the specification as an embodiment of the present invention. Thus, the claims are a further description and are an addition to the embodiments of the present invention. The discussion of a reference in the Description of Related Art is not an admission that it is prior art to the present invention, especially any reference that may have a publication date after the priority date of this application. The disclosures of all patents, patent applications, and publications cited herein are hereby incorporated by reference, to the extent that they provide exemplary, procedural or other details supplementary to those set forth herein.

While embodiments of the invention have been shown and described, modifications thereof can be made by one skilled in the art without departing from the spirit and teachings of the invention. Many variations and modifications of the invention disclosed herein are possible and are within the scope of the invention. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.). For example, whenever a numerical range with a lower limit, R_{L}, and an upper limit, Ru, is disclosed, any number falling within the range is specifically disclosed. In particular, the following numbers within the range are specifically disclosed: R=R_{L}+k*(Ru-R_{L}), wherein k is a variable ranging from 1 percent to 100 percent with a 1 percent increment, i.e., k is 1 percent, 2 percent, 3 percent, 4 percent, 5 percent, . . . , 50 percent, 51 percent, 52 percent, . . . , 95 percent, 96 percent, 97 percent, 98 percent, 99 percent, or 100 percent. Moreover, any numerical range defined by two R numbers as defined above is also specifically disclosed. Use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, etc.

What is claimed is:

1. A composition comprising:
a metal-organic framework (MOF) nanoparticle for oral protein administration, wherein the MOF comprises a nano-scale acid-resistant MOF;
a small molecular protein/polypeptide drug and a targeting molecule, wherein the MOF is loaded with the small molecular protein/polypeptide; and
targeting molecules, wherein a surface of the MOF is coated with the targeting molecules.

2. The composition of claim 1, wherein: the MOF is UiO-68-NH₂, PCN-222, PCN-224, or any combination thereof.

3. The composition of claim 1, wherein: the small molecular protein/polypeptide is a protein/polypeptide with a molecular weight less than or equal to about 10,000 Dalton and has therapeutic effect comprising salmon calcitonin, glucagon, interferon, or growth hormone.

4. The composition of claim 1, wherein: the targeting molecules can target receptors on intestinal epithelial cell membranes, comprising transferrin, neonatal Fc receptor protein, cell penetrating peptide, octaarginine, or any combination thereof.

5. The composition of claim 1, wherein: the MOF is UiO-68-NH₂, the small molecular protein/polypeptide is insulin, and the targeting molecules are transferrin.

6. The composition of claim 5, wherein: the MOF is UiO-68-NH₂, the mass ratio of the insulin to the MOF is about 1:1-about 1:6 UiO-68-NH₂, and the mass ratio of the transferrin to the MOF is about 1:0.1-about 1:1.

7. A method of preparing metal organic framework (MOF) nanoparticles for oral protein administration according to claim 1, the method comprising:
adding the micromolecule protein/polypeptide or the solution thereof into the acid-resistant metal organic framework water solution;
continuously stirring to ensure that the acid-resistant metal organic framework is coated with the micromolecule protein/polypeptide; and
adding the targeting molecules or the solution thereof, and continuously stirring to ensure that the targeting molecule is adhered to the acid-resistant metal organic framework to obtain the metal organic framework nano-particle for oral protein administration.

8. The method of claim 7, wherein: the preparation method is carried out at about 4-about 35° C.

9. The method of claim 7, wherein: the acid-resistant metal organic framework is UiO-68-NH₂ when the small molecular protein/polypeptide is insulin and the targeting molecule is transferrin, the preparation method comprises the following steps:
dissolving insulin powder in dilute hydrochloric acid solution with a pH of about 1.0-about 3.0, and dropwise adding the insulin hydrochloric acid solution to UiO-68-NH₂ under stirring at room temperature;
adding the mixture into the aqueous solution and continuously stirring for about 0.1 to about 12 hours; and
under the condition of stirring at room temperature, adding the transferrin solution and continuously stirring for about 0.1-about 12 hours, wherein the unencapsulated or unattached proteins are removed by centrifugation.

10. A composition comprising:
a metal-organic framework (MOF) nanoparticle for oral protein administration, wherein the MOF comprises a nano-scale acid-resistant MOF;
a small molecular protein/polypeptide drug and a targeting molecule, wherein the MOF is loaded with the small molecular protein/polypeptide; and
targeting molecules, wherein a surface of the MOF is coated with the targeting molecules.

11. The composition of claim 10, wherein the MOF nanoparticle comprises a conjugated mesoporous acid-resistant MOF.

12. The composition of claim 10, wherein the MOF comprises a Zr-MOF.

13. The composition of claim 10, wherein: the MOF is PCN-777.

14. The composition of claim 10, wherein: the small molecular protein/polypeptide is a protein/polypeptide with a molecular weight less than or equal to about 10,000 Dalton and has therapeutic effect comprising salmon calcitonin, glucagon, interferon, or growth hormone.

15. The composition of claim 10, wherein: the targeting molecules can target receptors on intestinal epithelial cell membranes, comprising transferrin, neonatal Fc receptor protein, cell penetrating peptide, octaarginine, or any combination thereof.

16. The composition of claim 10, wherein: the MOF is PCN-777, the small molecular protein/polypeptide is insulin, and the targeting molecules comprise a folate.

17. The composition of claim 10, wherein the targeting molecules comprise folic acid.

18. The composition of claim 10, wherein: the MOF is PCN-777, the targeting molecule is folic acid, and the mass ratio of the folic acid to the PCN-777 is between about 1:10 to about 4:10.

19. A method of preparing metal organic framework (MOF) nanoparticles for oral protein administration, the method comprising:
adding the micromolecule protein/polypeptide or the solution thereof into an acid-resistant metal organic framework solution;
continuously stirring to ensure that the acid-resistant metal organic framework is coated with the micromolecule protein/polypeptide; and
adding the targeting molecules or the solution thereof, and continuously stirring to ensure that the targeting molecule is adhered to the acid-resistant metal organic framework to obtain the metal organic framework nano-particle for oral protein administration.

20. The method of claim 19, wherein: the preparation method is carried out at about 80-about 130° C.
